SEQUENCE LISTING (64 sequences)

SEQ ID NO: 1 - DNA, 1149 bp, Escherichia coli
atgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctaa

SEQ ID NO: 2 - protein, 382 aa, Escherichia coli
MSLKEKTQSLFANAFGYPATHTIQAPGRVNLIGEHTDYNDGFVLPCAIDYQTVISCAPRDDRKVRVMAADYENQLDEFSLDAPIVAHENYQWANYVRGVVKHLQLRNNSFGGVDMVISGNVPQGAGLSSSASLEVAVGTVLQQLYHLPLDGAQIALNGQEAENQFVGCNCGIMDQLISALGKKDHALLIDCRSLGTKAVSMPKGVAVVIINSNFKRTLVGSEYNTRREQCETGARFFQQPALRDVTIEEFNAVAHELDPIVAKRVRHILTENARTVEAASALEQGDLKRMGELMAESHASMRDDFEITVPQIDTLVEIVKAVIGDKGGVRMTGGGFGGCIVALIPEELVPAVQQAVAEQYEAKTGIKETFYVCKPSQGAGQC

SEQ ID NO: 3 - DNA, 1383 bp, Glycine max
atggcgacgcacgaggagcttccgatcccgatttacaacaacctagaacctgtctatggcggaagttccgcactcgaagaagctcagcttcgtttcgacattttgaagtccaaattcatcgatatcttcggccatcatcctcaaatctttgctcgctcacccgggagagtgaacttgattggggagcacattgattatgaaggttattcggtgctgcctatggcaattcggcaagacacgatcgtggcgattcggaaaaatgaggcggaaaaggttctcaagatagctaacgtgaatggtgaaaaatattcactttgtacttatcccgccgatcctctccaggaaatcgacttgaagaaccacaaatggggacattattttatttgtgggtacaaaggtttccatgactatgcaaaattgaaaggagtggatgttggcaaacctgttggacttgaagttcttgttgatggaacagtgccgacaggttctggactatcaagctctgcagcatttgtctgctcatccacaattgctattatggctgcttttgatgtgaacttcccgaagaaagaacttgcacaagttacatgtgattgtgaacgacatattgggactcaatctggtgggatggatcaggcaatctctgtcatggccaagactgggtttgcagaactgattgatttcaacccaattcgtgcaacagatgtgcaacttcctgctggtgggacttttgtgatagctcattctttggcagagtctcaaaaggctgttactgctgccacaaattataataatagggttgtcgaatgccgtttggcttctattgtgctcgctataaagctagggatggatccaaaagaggcaatatcaaaagtgagcacactgtctgatgttgaagggttatgtgtatcatttgctggtatttataactcatctgatcctgtacttgctgtaaaggaatatttgaaggaagaaccatatacagctgaagaaattgaagcagttacaggggaaaagctgacttcatttttgaacaataatgcagcttatttagaagtgttaaaagttgcaaagcaatacaagttgcatcagagagctgctcatgtgtattcagaagccaagagggttcatgctttcaaggatgtcgtatcttcgaatctaagtgacgaggacatgctaaagaagcttggtgaccttatgaacgagagtcatcatagctgcagcgttttatatgaatgcagctgtccggagttggaagaacttgtaaatatttgtcgtacaatggtgctcttggagcaaggcttactggacctggatggggtggttgtgctgttgctttggtggaaaaagaggataagttccccaatttattccttagtttga

SEQ ID NO: 4 - protein, 460 aa, Glycine max
MATHEELPIPIYNNLEPVYGGSSALEEAQLRFDILKSKFIDIFGHHPQIFARSPGRVNLIGEHIDYEGYSVLPMAIRQDTIVAIRKNEAEKVLKIANVNGEKYSLCTYPADPLQEIDLKNHKWGHYFICGYKGFHDYAKLKGVDVGKPVGLEVLVDGTVPTGSGLSSSAAFVCSSTIAIMAAFDVNFPKKELAQVTCDCERHIGTQSGGMDQAISVMAKTGFAELIDFNPIRATDVQLPAGGTFVIAHSLAESQKAVTAATNYNNRVVECRLASIVLAIKLGMDPKEAISKVSTLSDVEGLCVSFAGIYNSSDPVLAVKEYLKEEPYTAEEIEAVTGEKLTSFLNNNAAYLEVLKVAKQYKLHQRAAHVYSEAKRVHAFKDVVSSNLSDEDMLKKLGDLMNESHHSCSVLYECSCPELEELVNICRTMVLLEQGLLDLDGVVVLLLWWKKRISSPIYSLV

SEQ ID NO: 5 - DNA, 1296 bp, Glycine max
atggcttcgcggtgctggccttctgatgctgagctaaatgaattgagagagagagtttcgaaaatagtggatctgaataaagaggaagttcgagttgtggtatctccctatcgaatatgtcctttgggggcacacattgatcatcagggtgggaccgttgcagctatgacaatcaataagggaatacttctggggtttgctccttctggcagtaatcaggttgtaattcgttctggacagtttgagggagaagttaagttcagagttgatgagattcagcagccaaaagataagagtttggacaaagattcatcagagctacaggaacaatgtaactgggggcgttatgctagaggagccgtatatgcactacagagtagaggaaacaatctttctaagggtatcattggatacatatgtggttctgaaggtctggacagttcgggtttaagctcttctgctgcagttggagtggcttacctcatggctttgcaatatgcaaatgatttagtaatatctcccacagaaattattgaatatgataggttgattgagaatgaatatttgggtctgaaaaatggcataatggaccaatcagctattttgctttcaagccatggttgtttgatgtgcatgaattgcaagaccaaagattataaacttgtttaccaaccaaaggttctagaatacaacgagagtgggcagccgaaagcaaccagaatattgttggcactttcggggttgaagcaagctttgatgaataaccctggatataacaagcgagttgtagagtgtcgagaggccgcacaaattcttattgaagcatctggagattacaaaacagagcccatcctatctaacgttgatccagaagtatatgacactcacaagcacaaattagaacctgatctagccaaaagagcagagcattatttctctgagaatatgcgagttatgaaaggagttgaggcttgggcgatgggcaatttaaaagattttggaatgcttattacagcttctggtcggagttccattcaaaattatgaatgtggttgtgaaccactgattcaactgtatgagatccttttgagggctcccggtgtattgggagcgcgcttcagtggtgctgggtttagagggtgttgccttgcatttgtggaggctgaccttgcaactgaagctgcatcatttgtcaggagtgaatatctcaaggtacagccagagttagcaagccaaataagcaaagacactgcagttttgatatgtgaatctggtgattgtgcacgtgtaatttaa

SEQ ID NO: 6 - protein, 431 aa, Glycine max
MASRCWPSDAELNELRERVSKIVDLNKEEVRVVVSPYRICPLGAHIDHQGGTVAAMTINKGILLGFAPSGSNQVVIRSGQFEGEVKFRVDEIQQPKDKSLDKDSSELQEQCNWGRYARGAVYALQSRGNNLSKGIIGYICGSEGLDSSGLSSSAAVGVAYLMALQYANDLVISPTEIIEYDRLIENEYLGLKNGIMDQSAILLSSHGCLMCMNCKTKDYKLVYQPKVLEYNESGQPKATRILLALSGLKQALMNNPGYNKRVVECREAAQILIEASGDYKTEPILSNVDPEVYDTHKHKLEPDLAKRAEHYFSENMRVMKGVEAWAMGNLKDFGMLITASGRSSIQNYECGCEPLIQLYEILLRAPGVLGARFSGAGFRGCCLAFVEADLATEAASFVRSEYLKVQPELASQISKDTAVLICESGDCARVI

SEQ ID NO: 7 - DNA, 1515 bp, Zea mays
atggccgcggtacccgccagcgcccccgccgccgccgaggcggcggagctcgtgccgacgctctcctcgctggagccggtctacggcgcgggcgcgcagctcgacgagtcgcgcctccgcttcgcccgcctcggggaccgcttccacgccgtctacggcgcccgccccgcgctcttcgcccgctccccagggagggtcaatctgatcggggagcacatcgactacgagggctactcggtgttgccgatggccatccgccaggacatgatcgtcgccatccgaagggccaacggcgcccaggtgagggtcgccaacgtcgacgacaagtaccccctctgcgtttaccccgccgacccagacaaggaaattgacgtaaaaaatcacaaatgggggcactatttcatgtgtggatacaagggagtttatgaatactgtagatcaaaagggatagatctgggcaaacctgttgcgcttgacgtcgttgttgatggcacagttcctcaaggctctggactgtcaagctcagcagcatttgtctgttcagcaacaattgctatcatgggaatccttgacaaaaactttccaaagaaagaagttgctcaattcacttgtctgtctgagcgccacattggaacgcaatctggcggcatggatcaggctatatctatcatggcgaaacctggattcgctgagttgatagattttaatccaatccatgcaactgacgtccaactaccttcaggtggtacatttgtgatcgcccattgtttggccgagtccaagaaagcagagacagctgcaacaaattataataaccgtgttgtggagtgtcgcttagcagcgattgttcttgccatcaaacttgggatggataggaaaaaggctatctcctccgttacaaccctctccgatgttgaggggctatgtgtttcttttgctgggagagaaggttcatctgatcctgcagtagctgtgaagaaacttctgcatgaggacccatatacagctggagaaatagagaaaattacaggagaaggcctgacatctgtcttccagggctctcagacgtcattggatgttataaaagcggcaaagcactacaagctatttcagcgtgcgacccatgtctactctgaagcaaggagggtttatgctttcagggatactgtctcttcaaaactcagtgaggaaggcaagcttaagaaacttggtgatcttatgaacgagagccattacagctgcagcgtgctatacgaatgcagttgccctgagctggaggagcttgtgaaggtctgcagagacaacggagcactgggagcacgtctgacaggagctgggtggggtggctgcgcggttgctctggtcaaggagcccatcgtccctcagttcattctgaaactaaaggaaatgtactacaaatcaaggattgacaggggagtaatcaagcagggcgatgtgggcctatacgtgttcgcatccaagccgtcgagcggcgcggccatactgaggttgtag

SEQ ID NO: 8 - protein, 504 aa, Zea mays
MAAVPASAPAAAEAAELVPTLSSLEPVYGAGAQLDESRLRFARLGDRFHAVYGARPALFARSPGRVNLIGEHIDYEGYSVLPMAIRQDMIVAIRRANGAQVRVANVDDKYPLCVYPADPDKEIDVKNHKWGHYFMCGYKGVYEYCRSKGIDLGKPVALDVVVDGTVPQGSGLSSSAAFVCSATIAIMGILDKNFPKKEVAQFTCLSERHIGTQSGGMDQAISIMAKPGFAELIDFNPIHATDVQLPSGGTFVIAHCLAESKKAETAATNYNNRVVECRLAAIVLAIKLGMDRKKAISSVTTLSDVEGLCVSFAGREGSSDPAVAVKKLLHEDPYTAGEIEKITGEGLTSVFQGSQTSLDVIKAAKHYKLFQRATHVYSEARRVYAFRDTVSSKLSEEGKLKKLGDLMNESHYSCSVLYECSCPELEELVKVCRDNGALGARLTGAGWGGCAVALVKEPIVPQFILKLKEMYYKSRIDRGVIKQGDVGLYVFASKPSSGAAILRL

SEQ ID NO: 9 - DNA, 951 bp, Escherichia coli
atgaacgagttagacggcatcaaacagttcaccactgtcgtggcagacagcggcgatattgagtccattcgccattatcatccccaggatgccaccaccaatccttcgctgttactcaaagctgccggattatcacaatatgagcatttaatagacgatgctatcgcctggggtaaaaaaaatggcaagacccaggaacaacaggtggtcgcagcgtgtgacaaactggcggtcaatttcggtgctgaaatcctcaaaatcgtacccggtcgcgtgtcaacagaagttgatgcacgcctctcttttgataaagaaaagagtattgagaaggcgcgccatctggtggacttgtatcagcaacaaggcgttgagaaatcacgcattctgatcaagctggcttcgacctgggaaggaattcgcgcggcagaagagctggaaaaagaaggtattaactgcaacctgacgctgctgttttcttttgcacaggcacgggcctgtgcggaagcaggcgtttttctgatttcgccgtttgtcgggcgtatttatgactggtatcaggcacgcaagccgatggacccgtatgtggtggaagaagatccgggcgttaaatcggtgcgcaatatctacgactactataagcaacaccactatgaaaccattgtgatgggcgcgagcttccgtcgcaccgaacaaatcctcgccttaaccggctgcgatcgactgactatcgcaccgaatttactgaaggagctgcaggaaaaagtttcgccagtggtacgtaaattaatcccaccttctcagacgttcccacgcccagctcccatgagcgaagcggagttccgttgggagcacaatcaggatgcgatggcggtagaaaaactgtctgaaggcattcgtctgttcgccgttgatcaacgcaaactggaagatcttcttgccgccaaactataa

SEQ ID NO: 10 - protein, 316 aa, Escherichia coli
MNELDGIKQFTTVVADSGDIESIRHYHPQDATTNPSLLLKAAGLSQYEHLIDDAIAWGKKNGKTQEQQVVAACDKLAVNFGAEILKIVPGRVSTEVDARLSFDKEKSIEKARHLVDLYQQQGVEKSRILIKLASTWEGIRAAEELEKEGINCNLTLLFSFAQARACAEAGVFLISPFVGRIYDWYQARKPMDPYVVEEDPGVKSVRNIYDYYKQHHYETIVMGASFRRTEQILALTGCDRLTIAPNLLKELQEKVSPVVRKLIPPSQTFPRPAPMSEAEFRWEHNQDAMAVEKLSEGIRLFAVDQRKLEDLLAAKL

SEQ ID NO: 11 - DNA, 855 bp, Brassica napus
atggctttggcggactctacctgtgcgggccttgataccactgagtccaaactctcttgctttttcgataaggctattgtgaatgtaggtggagatcttgtcaaactcgttccaggtcgtgtttcgactgaagtggatgcacgtcttgcttatgacaccaacgccattatccgcaaggttcatgatctgttaagactctacaatgaaatcgatgtacctcacgaccggctgcttttcaaaatccctgcaacttggcaaggtattgaagctgcaaagctgctggaatccgagggaattcaaacgcacttgaccttcgtttacagctttggtgtaccaacagcagccgctcaagccggtgcctctgtcattcagattttcgtcggtcgcctcagggactgggcgcgcaatcattcaggagatgccgagattgaaactgcggttaaatccggggaagatccaggtttggccttggtcagaagatcgtataactacattcacaagtatggttacaagtccaagctaatggctgctgctgtcagaaacaaacaggacttgttcagtcttctcggggttgattatgtgattgcgccattgaaggtattgcaatctctcaaagactcaccagccgttcctgacgatgagaaatactcgttcgttcggaaactttctcctgagaccgcaacacactacaacttcaccaacaaagagctgatcaagtgggaccagctaagcttggcttcagctatgggtcctgcatcagtggagcttttatcagctggtgtggaaggttatgcgaaacaagcgaaacgcgttgaagagctctttgggaagatttggccacctccaaatgtctaa

SEQ ID NO: 12 - protein, 284 aa, Brassica napus
MALADSTCAGLDTTESKLSCFFDKAIVNVGGDLVKLVPGRVSTEVDARLAYDTNAIIRKVHDLLRLYNEIDVPHDRLLFKIPATWQGIEAAKLLESEGIQTHLTFVYSFGVPTAAAQAGASVIQIFVGRLRDWARNHSGDAEIETAVKSGEDPGLALVRRSYNYIHKYGYKSKLMAAAVRNKQDLFSLLGVDYVIAPLKVLQSLKDSPAVPDDEKYSFVRKLSPETATHYNFTNKELIKWDQLSLASAMGPASVELLSAGVEGYAKQAKRVEELFGKIWPPPNV

SEQ ID NO: 13 - DNA, 852 bp, Glycine max
atggctttagctgattctgagtgttatggacttgaaaatcctaacgcgcgattgtcttgttttgtcaacaaggctttcgcgaatatcggtagtgacatggcaaagcttgtccctggccgtgtttcgacagaagtggatgcgcggcttgcttatgacacacatgccattatcaggaaggtgcatgacctgttgaagttgtacaatgatagcaatgtacctccgcaacgtctgttgtttaaaattccttccacttggcaaggaatagaggctgcaaggttgctggaatccgaaggcatacagacacatttgacctttgtctacagttttgctcaagctgcagctgcagctcaagctggtgcttctgtcattcaaatttttgttggtcgcataagggattgggcacgtaatcattctggtgacacagagatagaatctgctcagctaagaggagaggatccagggttggcattggtgacaaaagcttacaattatattcacaaatatggacataagtcaaagttgatggcagcagcagttcgcaacaaacaggacctatttagtcttctgggggttgactatatcatagctcctttaaaggtattgcagtctctcaaagaatctgttgcttctcctgatgagaagtactcttttgttaggaggttatcccctcagtctgctgccaagtacacatttagtgatgaagagcttgttagatgggacgaagatagcctctcaaaggccatggggcctgcagctgtgcagcttctggctgctggactggatggccatgctgatcaagcaaagcgggtggaagagttatttgggaaaatttggccaccaccaaatgtatga

SEQ ID NO: 14 - protein, 283 aa, Glycine max
MALADSECYGLENPNARLSCFVNKAFANIGSDMAKLVPGRVSTEVDARLAYDTHAIIRKVHDLLKLYNDSNVPPQRLLFKIPSTWQGIEAARLLESEGIQTHLTFVYSFAQAAAAAQAGASVIQIFVGRIRDWARNHSGDTEIESAQLRGEDPGLALVTKAYNYIHKYGHKSKLMAAAVRNKQDLFSLLGVDYIIAPLKVLQSLKESVASPDEKYSFVRRLSPQSAAKYTFSDEELVRWDEDSLSKAMGPAAVQLLAAGLDGHADQAKRVEELFGKIWPPPNV

SEQ ID NO: 15 - DNA, 249 bp, Escherichia coli
atgtgtattggcgttccaggccaggtgctggctgtcggtgaagatattcaccagcttgcgcaggttgaagtatgtggtatcaagcgcgatgtgaatatcgccctgatttgtgaaggtaaccctgccgatctactgggccagtgggtgctggtacacgtcggatttgccatgagcatcatcgacgaagatgaagccaaagccacattagacgcactgcgccaaatggattacgacattaccagcgcgtga

SEQ ID NO: 16 - protein, 82 aa, Escherichia coli
MCIGVPGQVLAVGEDIHQLAQVEVCGIKRDVNIALICEGNPADLLGQWVLVHVGFAMSIIDEDEAKATLDALRQMDYDITSA

SEQ ID NO: 17 - DNA, 1674 bp, Saccharomyces cerevisiae
atgcctatccccgttggaaatacgaagaacgattttgcagctttacaagcaaaactagatgcagatgctgccgaaattgagaaatggtggtctgactcacgttggagtaagactaagagaaattattcagccagagatattgctgttagacgcgggacattcccaccaatcgaatacccatcttcggtcatggccagaaaattattcaaggtattagagaagcatcacaatgagggtacagtctctaaaactttcggtgccctagatcctgtccagatttctcaaatggcaaaatacttagacacaatctatatttctggttggcagtgttcatcaactgcttccacctcaaatgaacctggtccagacttagctgattatccaatggacaccgttccaaacaaagtggaacatttgttcaaggcccaattgtttcacgacagaaaacaactagaggcacggtcaaaggctaaatctcaggaagaactcgatgagatgggtgccccaattgactacctaacaccaattgtcgctgatgcagacgcaggccacggcggtttaaccgcagtcttcaaattgaccaagatgttcattgagcgtggtgctgctgggatccacatggaagaccagacatctacaaataagaaatgtgggcatatggcaggaagatgtgttatacccgttcaggaacatgttaacagattggtgactattagaatgtgtgctgatatcatgcattctgacttaattgtcgttgctaggactgattcagaagcagccactttgattagctcaaccatcgataccagagatcattatttcattgtcggtgccaccaatccaaatatcgagccatttgccgaagttttaaatgatgccatcatgagtggtgcatcaggacaagaactagctgacattgaacaaaaatggtgtagagacgctggactcaagttattccatgaagccgtcattgatgaaattgaaagatcagccctgtcaaataagcaagaattgattaagaaattcacctctaaagtgggtccattgactgaaacatcccacagagaagccaagaagctcgctaaagaaattcttggccacgaaattttcttcgactgggagctaccacgcgtaagggaagggttgtaccgttacagaggtgggacgcaatgttctatcatgagggcccgtgcatttgctccatatgctgatttggtatggatggaatctaactacccagacttccaacaggccaaggagtttgcagaaggtgttaaagagaaattccctgaccaatggctagcttacaacttgtctccatcctttaactggccaaaagccatgtccgttgatgaacaacacaccttcatccaaaggctgggtgatctaggttacatctggcaatttatcacattggccggtttacacactaacgctttagctgtccataacttctctcgtgactttgccaaggatgggatgaaagcttatgcccagaatgttcagcagagggaaatggacgatggtgttgatgtgttgaaacatcaaaaatggtctggtgcggagtacatcgatgggttattgaagttagctcaaggtggtgttagcgcaacagctgctatgggaaccggtgtcacagaagatcaattcaaagaaaatggcgtaaagaaatag

SEQ ID NO: 18 - protein, 557 aa, Saccharomyces cerevisiae
MPIPVGNTKNDFAALQAKLDADAAEIEKWWSDSRWSKTKRNYSARDIAVRRGTFPPIEYPSSVMARKLFKVLEKHHNEGTVSKTFGALDPVQISQMAKYLDTIYISGWQCSSTASTSNEPGPDLADYPMDTVPNKVEHLFKAQLFHDRKQLEARSKAKSQEELDEMGAPIDYLTPIVADADAGHGGLTAVFKLTKMFIERGAAGIHMEDQTSTNKKCGHMAGRCVIPVQEHVNRLVTIRMCADIMHSDLIVVARTDSEAATLISSTIDTRDHYFIVGATNPNIEPFAEVLNDAIMSGASGQELADIEQKWCRDAGLKLFHEAVIDEIERSALSNKQELIKKFTSKVGPLTETSHREAKKLAKEILGHEIFFDWELPRVREGLYRYRGGTQCSIMRARAFAPYADLVWMESNYPDFQQAKEFAEGVKEKFPDQWLAYNLSPSFNWPKAMSVDEQHTFIQRLGDLGYIWQFITLAGLHTNALAVHNFSRDFAKDGMKAYAQNVQQREMDDGVDVLKHQKWSGAEYIDGLLKLAQGGVSATAAMGTGVTEDQFKENGVKK

SEQ ID NO: 19 - DNA, 492 bp, Saccharomyces cerevisiae
atgtcagaattctataagctagcacctgttgacaagaaaggccaaccattccccttcgaccaattaaagggaaaagtggtgcttatcgttaatgttgcctccaaatgtggattcactcctcaatacaaagaactagaggccttgtacaaacgttataaggacgaaggatttaccatcatcgggttcccatgcaaccagtttggccaccaagaacctggctctgatgaagaaattgcccagttctgccaactgaactatggcgtgactttccccattatgaaaaaaattgacgttaatggtggcaatgaggaccctgtttacaagtttttgaagagccaaaaatccggtatgttgggcttgagaggtatcaaatggaattttgaaaaattcttagtcgataaaaagggtaaagtgtacgaaagatactcttcactaaccaaaccttcttcgttgtccgaaaccatcgaagaacttttgaaagaggtggaatag

SEQ ID NO: 20 - protein, 163 aa, Saccharomyces cerevisiae
MSEFYKLAPVDKKGQPFPFDQLKGKVVLIVNVASKCGFTPQYKELEALYKRYKDEGFTIIGFPCNQFGHQEPGSDEEIAQFCQLNYGVTFPIMKKIDVNGGNEDPVYKFLKSQKSGMLGLRGIKWNFEKFLVDKKGKVYERYSSLTKPSSLSETIEELLKEVE

SEQ ID NO: 21 - DNA, 510 bp, Brassica napus
atggctgcttcctccgaacccaaatccatctatgatttcaccgtcaaggatgcgaagggaaacgatgttgatctaagcacttacaaggggaaggttctgttgattgtcaacgttgcttctcagtgtggcttgaccaattcgaactatactgagcttgcacagctgtaccagaagtacaaagaccatgggtttgagatccttgcattcccctgtaaccagtttggtaatcaagaacctggttctaatgaagagattgttcagtttgcttgtacccgtttcaaggccgagtaccccatcttcgacaaggttgatgtgaacggtgactcggctgctccaatctataagttcctgaaatcaagcaaaggagggctttttggagacggaatcaagtggaacttcgccaagttcttggttgacaaagatgggaatgttgtggaccgttacgctccaactacttcccctctcagcattgagaaggacctgaagaaactgttgggagttactgcttaa

SEQ ID NO: 22 - protein, 169 aa, Brassica napus
MAASSEPKSIYDFTVKDAKGNDVDLSTYKGKVLLIVNVASQCGLTNSNYTELAQLYQKYKDHGFEILAFPCNQFGNQEPGSNEEIVQFACTRFKAEYPIFDKVDVNGDSAAPIYKFLKSSKGGLFGDGIKWNFAKFLVDKDGNVVDRYAPTTSPLSIEKDLKKLLGVTA

SEQ ID NO: 23 - DNA, 606 bp, Brassica napus
atgcacgcggcggcggataatacagcaacaaaaaacagacgtagaacttggttggagagttccggctgcgagaagatgggtggttcaatatcagtgcctgaaaaatctatccatgaattcactgtcaaggatagctccggcaaggaggttgaccttagcgtttaccaagggaaggttcttctcatcgtcaacgtcgcttctaaatgcggtttcactcaaaccaattacacccagcttactgaactttaccggaaatacaaagatcaagggttggtgatattggcgtttccttgcaaccagtttttgaaccaagagcctggcactagccaagatgctcatgaatttgcttgtactaggtttaaggccgagtatcctgtcttccaaaaggtgcgtgtgaatggtcaaaacgcagcaccagtctacaaattcctcaagtcaaagaaaccatctttccttggaagcaggatcaaatggaacttcaccaagttcttggtcggcaaagatggtcaagtcattgatcgttatggccccactgttccacctctttccatcgagaaagacatcaagaaagccctcggagatgaaggagcgtttccaagtacttag

SEQ ID NO: 24 - protein, 201 aa, Brassica napus
MHAAADNTATKNRRRTWLESSGCEKMGGSISVPEKSIHEFTVKDSSGKEVDLSVYQGKVLLIVNVASKCGFTQTNYTQLTELYRKYKDQGLVILAFPCNQFLNQEPGTSQDAHEFACTRFKAEYPVFQKVRVNGQNAAPVYKFLKSKKPSFLGSRIKWNFTKFLVGKDGQVIDRYGPTVPPLSIEKDIKKALGDEGAFPST

SEQ ID NO: 25 - DNA, 510 bp, Brassica napus
atggcggaggaatctccacagtctatctacgacttcaccgttaaggatattgaaggtaaagatgtgagtttgagccaattcaaaggcaaaactcttttgattgtaaacgttgcctccaaatgtggtctgacggatgcaaactacaaggaactgaatgttctatacgataaatacaaggaccaagggttggagattttagcgtttccgtgcaatcagttcttgggacaagaaccaggaaacaatgaagagatccaacaaactgtctgcacaaagttcaaagctgagttccccatctttgacaaggtggatgtgaacgggaagaacacggcgccattatacaaatacttgaaagcagagaaaggaggtttgcttattgacgcaatcaaatggaacttcacaaagttcttggtttctcctgacggcaaagtctcccagagatattctcctagaacgtctcctcttcaattcgagaaagacattcaagctctgttgggacaggcttcatcttga

SEQ ID NO: 26 - protein, 169 aa, Brassica napus
MAEESPQSIYDFTVKDIEGKDVSLSQFKGKTLLIVNVASKCGLTDANYKELNVLYDKYKDQGLEILAFPCNQFLGQEPGNNEEIQQTVCTKFKAEFPIFDKVDVNGKNTAPLYKYLKAEKGGLLIDAIKWNFTKFLVSPDGKVSQRYSPRTSPLQFEKDIQALLGQASS

SEQ ID NO: 27 - DNA, 501 bp, Glycine max
atggccacctcaaacgccaaatcattccatgatttcaccgttatagatgccaagggaaatgatattaaccttggtgactacaaaggaaaggtccttatcattgtcaatgttgcctcacaatgtggcttgactaattcaaattacactgagctcagtcagttgtatgagaaatacaaacagaaaggtctggaaattctggcgttcccatgcaatcagtttggggcacaggagcctggatctaatgaacagatacaagagtttgtttgtactcgcttcaaggctgagtttcccgtttttgacaaggttgatgtgaatggtgacaaggctgctccactgtacaaatatctaaaatcaagcaaaggtggactccttggggatggcatcaaatggaacttcgccaagttccttgttgataaagaggggaatgttgttgatcgctatgcacccacaacttctcctctgagcattgagaaggacttgctgaagttgttggatgcatga

SEQ ID NO: 28 - protein, 166 aa, Glycine max
MATSNAKSFHDFTVIDAKGNDINLGDYKGKVLIIVNVASQCGLTNSNYTELSQLYEKYKQKGLEILAFPCNQFGAQEPGSNEQIQEFVCTRFKAEFPVFDKVDVNGDKAAPLYKYLKSSKGGLLGDGIKWNFAKFLVDKEGNVVDRYAPTTSPLSIEKDLLKLLDA

SEQ ID NO: 29 - DNA, 501 bp, Glycine max
atggccacctcaagcgccaaatcagtccatgatttcaccgttaaagatgccaagggaaatgatattaatcttggtgactacaaaggaaaggtccttatcattgtcaatgttgcctcacaatgtggcttgactaattcaaattacactgagctcagtcagttgtatgagaaatacaaacagaaaggtctggaaattctggcatttccatgcaatcagtttggggcacaggagcctggatctaatgaacagatacaagagtttgtttgtactcgcttcaaggctgagtttcccgtttttgacaaggttgatgtgaatggtgacaaagctgctccactgtacaagtatctaaaatcaagcaaaggtggactctttggggatggtatcaaatggaacttctccaagttccttgttgataaagagggaaatgtggttgatcgctatgcacccacaacttctcctctgagcattgagaaggacttgctgaagttgttggatgcatga

SEQ ID NO: 30 - protein, 166 aa, Glycine max
MATSSAKSVHDFTVKDAKGNDINLGDYKGKVLIIVNVASQCGLTNSNYTELSQLYEKYKQKGLEILAFPCNQFGAQEPGSNEQIQEFVCTRFKAEFPVFDKVDVNGDKAAPLYKYLKSSKGGLFGDGIKWNFSKFLVDKEGNVVDRYAPTTSPLSIEKDLLKLLDA

SEQ ID NO: 31 - DNA, 513 bp, Glycine max
atgggtgcttcggcatcggtcacagaaaaatccatccatgaattcatggtcaaggatgctaagggcagagacgtgaacctcagcacctacaaagggaaggttcttcttgtagttaacgtcgcttcaaaatgtggatttacaaattccaattacacccagttaactgagctttacagcaaatataaagacagaggtcttgagatactggcatttccatgcaaccagtttctgaaacaagagcccgggagtagccaggaggcagaggaatttgcctgtacaaggtacaaggctgagtatcccatttttggaaaggtacgtgtcaatggacctgatacagcacctgtctacaaattccttaaagcaaataaaacaggatttctgggtagtaggataaagtggaatttcactaagtttttggttgacaaggaagggcatgtcctcgctcgttatggtccaaccacctcaccgttgtccattgaaaatgacatcaagacagcattgggggaggcttga

SEQ ID NO: 32 - protein, 170 aa, Glycine max
MGASASVTEKSIHEFMVKDAKGRDVNLSTYKGKVLLVVNVASKCGFTNSNYTQLTELYSKYKDRGLEILAFPCNQFLKQEPGSSQEAEEFACTRYKAEYPIFGKVRVNGPDTAPVYKFLKANKTGFLGSRIKWNFTKFLVDKEGHVLARYGPTTSPLSIENDIKTALGEA

SEQ ID NO: 33 - DNA, 513 bp, Glycine max
atgggtgcttcgctatcggtctcggaaaaatccatccatgaattcatggtcaaggatgctaagggcagagacgtgaacctcagcatctacaaagggaaggttcttcttgtagtaaatgtcgcttcaaaatgtggatttacgaataccaattacacccagttaactgagctttacagcaaatacaaagacagaggccttgagatactggcatttccatgcaaccagtttctgaagcaggagcctgggagtagccaggacgtagaggaatttgcctgcacaagatacaaggccgcgtatcccatttttggaaaggtacgtgtcaatggacctgatacagcacctgtctacaaattccttaaagcaaataaatcaggatttctgggttctaggataaagtggaatttcaccaagtttttggttgacaaggaagggaatgtcctccggcgttatggttcaaccacctcaccgttttccattgaaaatgacatcaagagagcattgtgggaggcttga

SEQ ID NO: 34 - protein, 170 aa, Glycine max
MGASLSVSEKSIHEFMVKDAKGRDVNLSIYKGKVLLVVNVASKCGFTNTNYTQLTELYSKYKDRGLEILAFPCNQFLKQEPGSSQDVEEFACTRYKAAYPIFGKVRVNGPDTAPVYKFLKANKSGFLGSRIKWNFTKFLVDKEGNVLRRYGSTTSPFSIENDIKRALWEA

SEQ ID NO: 35 - DNA, 558 bp, Helianthus annuus
atgtctcaacaacaacaaaccctcaaatccgttcaccatttcaccgtcaaggatattcgtggaaatgaggtgtcattgagctcttacaaggggaaggttcttttgattgttaatgttgcatctaaatgtggactaacggagtcgaactacaaagagttgaatatattgtaccaaaaatacaaagatcaagattttgaaatcttggcttttccatgcaaccagtttcttaggcaagagccaggaacaaatgaagaaattcaagagaccgtatgcacgagattcaaagccgagttcccgatatttgacaagattgatgtcaatggaaacaatgcagcacccctttacaagtttttaaaatccgagaaaggtggtttcttggttgatggcatgaaatggaacttcaccaagttcttggtgaacaaagaaggaaaagttatccaaagatacggtcctcgaaccccgcctctagaattcgagaaagatattcaagatctgttgagttcgtcatcatcttacgagacaagaaaagagataataaacaatgggatgtgcacttga

SEQ ID NO: 36 - protein, 185 aa, Helianthus annuus
MSQQQQTLKSVHHFTVKDIRGNEVSLSSYKGKVLLIVNVASKCGLTESNYKELNILYQKYKDQDFEILAFPCNQFLRQEPGTNEEIQETVCTRFKAEFPIFDKIDVNGNNAAPLYKFLKSEKGGFLVDGMKWNFTKFLVNKEGKVIQRYGPRTPPLEFEKDIQDLLSSSSSYETRKEIINNGMCT

SEQ ID NO: 37 - DNA, 531 bp, Hordeum vulgare
atgggggcggccgaatctgtgccggagacctccgtacacgaattcaccgttaaggattgcaacggcaaggaggtgtgcctggacacgtacaaggggaaggtcctcctcatcgtcaacgtcgcctccaaatgcgggttcacggagactaattacacgcagctgacggagctttatcagaagtacagggagaaagactttgagatattagcattcccctgcaaccagtttttgcgacaagagccaggcagtgaccagcagatccaagactttgcatgcacaagattcaaagctgaatatccagtttttcagaaggtgcgtgtaaatggcccagatgctgcgccgctttacaagtttctaaaagctagcaaacctggtttgtttggttcaagaatcaagtggaactttaccaagtttcttgttgacaagaatggaaaagtaatcaacagatacgcaactgctaccactccattttcattcgagaaagatatccagaaggcacttgaggaggaacctgccgactcgcagaagtag

SEQ ID NO: 38 - protein, 176 aa, Hordeum vulgare
MGAAESVPETSVHEFTVKDCNGKEVCLDTYKGKVLLIVNVASKCGFTETNYTQLTELYQKYREKDFEILAFPCNQFLRQEPGSDQQIQDFACTRFKAEYPVFQKVRVNGPDAAPLYKFLKASKPGLFGSRIKWNFTKFLVDKNGKVINRYATATTPFSFEKDIQKALEEEPADSQK

SEQ ID NO: 39 - DNA, 501 bp, Oryza sativa
atggctgaacaatcttccaactccatttacgatttcactgtcaaggacatcagtggaaatgatgtgagtctgaatgattacagcgggaaggttctactgattgtgaatgtcgcctctcaatgtggtttgacacagacaaattacaaagaattgaatgtattgtacgagaagtacaagaatcaaggatttgaaatcttggcatttccgtgcaaccagtttgctggacaggaaccaggaaacaatgaagaaattcaggaagtagtttgcacaaggttcaaggctgaatttcctatctttgataaggttgaagtcaatgggaagaatgcagtgccactttacaagtttttaaaggagcagaaagggggaatatttggtgatggtatcaagtggaacttcacaaagttcttagtaaacaaagaagggaaggttgtggacagatatgcacctaccacctcacctctgaaaatcgagaaagacatcgagaagctcgtgcaatcttga

SEQ ID NO: 40 - protein, 166 aa, Oryza sativa
MAEQSSNSIYDFTVKDISGNDVSLNDYSGKVLLIVNVASQCGLTQTNYKELNVLYEKYKNQGFEILAFPCNQFAGQEPGNNEEIQEVVCTRFKAEFPIFDKVEVNGKNAVPLYKFLKEQKGGIFGDGIKWNFTKFLVNKEGKVVDRYAPTTSPLKIEKDIEKLVQS

SEQ ID NO: 41 - DNA, 513 bp, Zea mays
atgggggcggccgaatccgtgccggagacctccatacacgaattcaccgtcaaggattgcaacggcaaggaagtgagcctggaaacctacaaggggaaggtcctccttgttgttaacgtcgcctccaaatgtgggttcacggagaccaattacacgcagctgacggagctttatcagaagtacagggacaaagacttcgagatattggcattcccttgcaatcagttcttgcgacaggagccaggtactgatcagcagatacaagactttgcttgcaccagatttaaagctgaatacccagtttttcagaaggtgcgcgtaaacggaccagatgcggcgccggtttacaagtttctgaaagctagcaagcctggtttgtttgggtcatcaaggatcaaatggaactttaccaagtttctggtggacaaagatgggaaggtcatcgagagatacggaacctcgacagctccaatggcaattgagaaggacatccagaaggcccttgaggaataa

SEQ ID NO: 42 - protein, 170 aa, Zea mays
MGAAESVPETSIHEFTVKDCNGKEVSLETYKGKVLLVVNVASKCGFTETNYTQLTELYQKYRDKDFEILAFPCNQFLRQEPGTDQQIQDFACTRFKAEYPVFQKVRVNGPDAAPVYKFLKASKPGLFGSSRIKWNFTKFLVDKDGKVIERYGTSTAPMAIEKDIQKALEE

SEQ ID NO: 43 - DNA, 549 bp, Zea mays
atgtttgccatgcaagcaggcggacttgaattcgaaaaagttgaagaaggagaagtcaaagcaacatcattgttcgagttatctggaaatgacttaatgacaaatgaagttgttccactcagcaacttcaaaggcaaggtatgtttggttgtcaatgtatcaagcaaatgtggattaactccaaagaattatccagagcttgaacagttgtacaagacatatggtccaagaggatttgttgtgttggcattcccaacaaatcaattcgcaaatcaagaaccaggtacaccagaggatatcagaaaattggttgatggatatggagtcacatttccaatgtttgctaaaactgatgttaatggcctaactgctcatccagtgttcaagttcttgaaacaaaaccttggtggagtacttggaagttcaatcaaatggaatttcaccaagttcttatgtgacagaaatggtaaaccagtcaagagatacatgccaaccacccaaccattgtcatttgttgctgatattgaagcacttcttgatcaagaatga

SEQ ID NO: 44 - protein, 182 aa, Zea mays
MFAMQAGGLEFEKVEEGEVKATSLFELSGNDLMTNEVVPLSNFKGKVCLVVNVSSKCGLTPKNYPELEQLYKTYGPRGFVVLAFPTNQFANQEPGTPEDIRKLVDGYGVTFPMFAKTDVNGLTAHPVFKFLKQNLGGVLGSSIKWNFTKFLCDRNGKPVKRYMPTTQPLSFVADIEALLDQE

SEQ ID NO: 45 - DNA, 1557 bp, Synechocystis sp.
atggcgggtaaaaccactggggttgtggcggcgggccatgcccaaacggcagaagcgggcaaatgtatgctcgaagaagggggcaatgccttcgatgcggcgatcgcctcggtgttggcggcctgcgtagtggaatcgagtttaacttccctggggggagggggttttctgctggcccagacggcggcgaagaagagttacctgttcgattttttctgccaaacaccccaagttaacccaggggaaaaagcagtggacttctaccccgttgccctcaattttggtggagcttggcaaacttttcacattggtaaaggggcgatcgccgtgccggggatggtggcgggattatttgcggcccataggaaactggggcagctacctttcaaagtattgattgaaccagcggtggcatatgcccgccagggatttaccctcaaccgtttcaatgactttaccaatggtctgctggagccaattctgacccaacaagaggaaggcagaaagttttacgctccccaaggaaaaattctccgccaaggggaaaaggcctatctgccccagtttgcggacgtgctggaacaattggctcgccatggcccggattggttttaccggggagagttaaccgagtgggtgctggaatctttgggggaagccagtgctctgaccgccaaggactgggccgactatcaggtggaaattcgtctacccctgcgggcccaatatcgccaacggcaactgttaaccaatcctccccccagtgccggaggcattctcattgcttttgctttgcagttgctagaaaaatacgatttgagccaatatcccctgggcagtgcggcacaaattcagctttttagccaagtgatggccctgagtaaccaagcccgtcggcaatatttagatggcaatctccactgtggggacattgaagcaaaatttctcggcggcgatcgcctggcctcggaactaggacaatcaaaatttatcaataaattgggtagcaccacccacatcagtgttttagacggagagggcaatgccgccagcttaaccagttccaatggggaggggtctgggcattttattcccggcacgggcattatgctgaacaacatgttgggggaagaagacctcaatccccagggcttttaccaatggccaccggggcaacgcctatcatcgatgatggcccccactatacttttagaccaggaacaaccccggctagttttggggtcagggggatcaaatcgcattcgtagtgccattttgcaggtggtctgtcatcacctagattaccaattacccttagccgaagcggtggggcgggaacgtattcattgggaggcccataaattagatttggagccaacctctgtagctgatattctggcccagttgcgatttgacgacggcacccagggtaccctttggacggaacaaaatatgttttttgggggagttcatggggttgccaccaccactgcggggaccatggaaggggttggagatccccggcgatcgggggccgtggcctacagtctggaataa

SEQ ID NO: 46 - protein, 518 aa, Synechocystis sp.
MAGKTTGVVAAGHAQTAEAGKCMLEEGGNAFDAAIASVLAACVVESSLTSLGGGGFLLAQTAAKKSYLFDFFCQTPQVNPGEKAVDFYPVALNFGGAWQTFHIGKGAIAVPGMVAGLFAAHRKLGQLPFKVLIEPAVAYARQGFTLNRFNDFTNGLLEPILTQQEEGRKFYAPQGKILRQGEKAYLPQFADVLEQLARHGPDWFYRGELTEWVLESLGEASALTAKDWADYQVEIRLPLRAQYRQRQLLTNPPPSAGGILIAFALQLLEKYDLSQYPLGSAAQIQLFSQVMALSNQARRQYLDGNLHCGDIEAKFLGGDRLASELGQSKFINKLGSTTHISVLDGEGNAASLTSSNGEGSGHFIPGTGIMLNNMLGEEDLNPQGFYQWPPGQRLSSMMAPTILLDQEQPRLVLGSGGSNRIRSAILQVVCHHLDYQLPLAEAVGRERIHWEAHKLDLEPTSVADILAQLRFDDGTQGTLWTEQNMFFGGVHGVATTTAGTMEGVGDPRRSGAVAYSLE

SEQ ID NO: 47 - DNA, 432 bp, Synechocystis sp.
atgtttgattttgatgccaccctgcccctgatggcattgcagttcgtggttctcgcgttcctgctcaatgctattttctacaagccaatgaataaggttttggatgagcgggctgattacattcgcaccaatgaagaggatgcccgggagcggttagccaaggccaaggcgattacccaggagtatgagcaacagattaccgatgcccgtcggcagtcccaagctgtgatcgctgatgcccaagctgaagctaggcgcttggcggcggaaaagattgcggaggcccaacgggaatcccaacggcaaaaggaaacggcggcgcaagaaattgaggcccaacggcagtcggctctgagttctttagaacaggaggtggcggccctgagtaatcagattttgcacaaattgttaggccctgaattgattaaataa

SEQ ID NO: 48 - protein, 143 aa, Synechocystis sp.
MFDFDATLPLMALQFVVLAFLLNAIFYKPMNKVLDERADYIRTNEEDARERLAKAKAITQEYEQQITDARRQSQAVIADAQAEARRLAAEKIAEAQRESQRQKETAAQEIEAQRQSALSSLEQEVAALSNQILHKLLGPELIK

SEQ ID NO: 49 - DNA, 648 bp, Glycine max
atggcaaacatgattatggcttccacaaaacctctggttccagtctgcaccagttcccgttcccccacaccaaaactccccattctccaaatttcactccccaaagccccaaccttgaaactgaaactcccaatttcaaagccccagatgctgtccctcctgggagggatagcaccactggtcttggccagaccctccctagcagaagaatttgagaaagcagcactctttgacttcaacctcaccctgcccataataatggtggagtttctgctgctgatggttgccttggacaagatatggttcaccccacttgggaaattcatggacgagagggacgcggcgatcagggagaagctgagcagcgtgaaggacacgtcggaggaggtgaagcagctggaggagaaggccaatgctgtcatggcggctgctcgagcggagattgcagcggcgctgaacaccatgaagaaggagacgcaggctgaggtggagcagaagattgctgaggggaggaagaaagtcgaggctgagctgcaggaggctctgtctagcttggagaatcaaaaggaagaaaccatcaagtcccttgattcccagattgcagctcttagccaggagattgttaataaggttcttcccactgcttaa

SEQ ID NO: 50 - protein, 215 aa, Glycine max
MANMIMASTKPLVPVCTSSRSPTPKLPILQISLPKAPTLKLKLPISKPQMLSLLGGIAPLVLARPSLAEEFEKAALFDFNLTLPIIMVEFLLLMVALDKIWFTPLGKFMDERDAAIREKLSSVKDTSEEVKQLEEKANAVMAAARAEIAAALNTMKKETQAEVEQKIAEGRKKVEAELQEALSSLENQKEETIKSLDSQIAALSQEIVNKVLPTA

SEQ ID NO: 51 - DNA, 1617 bp, Saccharomyces cerevisiae
atgagttctgtcgcagaaaatataatacaacatgccactcataattctacgctacaccaattggctaaagaccagccctctgtaggcgtcactactgccttcagtatcctggatacacttaagtctatgtcatatttgaaaatatttgctactttaatctgtattcttttggtttgggaccaagttgcatatcaaatcaagaaaggttccatcgcaggtccaaagtttaagttctggcccatcatcggtccatttttggaatccttagatccaaagtttgaagaatataaggctaagtgggcatccggtccactttcatgtgtttctattttccataaatttgttgttatcgcatctactagagacttggcaagaaagatcttgcaatcttccaaattcgtcaaaccttgcgttgtcgatgttgctgtgaagatcttaagaccttgcaattgggtttttttggacggtaaagctcatactgattacagaaaatcattaaacggtcttttcactaaacaagctttggctcaatacttaccttcattggaacaaatcatggataagtacatggataagtttgttcgtttatctaaggagaataactacgagccccaggtctttttccatgaaatgagagaaattctttgcgccttatcattgaactctttctgtggtaactatattaccgaagatcaagtcagaaagattgctgatgattactatttggttacagcagcattggaattagtcaacttcccaattattatcccttacactaaaacatggtatggtaagaaaactgcagacatggccatgaagattttcgaaaactgtgctcaaatggctaaggatcatattgctgcaggtggtaagccagtttgtgttatggatgcttggtgtaagttgatgcacgatgcaaagaatagtaacgatgatgattctagaatctaccacagagagtttactaacaaggaaatctccgaagctgttttcactttcttatttgcttctcaagatgcctcttcttctttagcttgttggttgttccaaattgttgctgaccgtccagatgtcttagctaagatcagagaagaacaattggctgttcgtaacaatgacatgtctaccgaattgaacttggatttgattgagaaaatgaagtacaccaatatggtcataaaagaaactttgcgttacagacctcctgtcttgatggttccatatgttgttaagaagaatttcccagtttcccctaactataccgcaccaaagggcgctatgttaattccaaccttatacccagctttacatgatcctgaagtttacgaaaatcctgatgagttcatccctgaaagatgggtagaaggctctaaggctagtgaagcaaagaagaattggttggtttttggttgtggtccacacgtttgcttaggtcaaacatatgtcatgattaccttcgccgctttgttgggtaaatttgcactatatactgatttccatcatacagtgactccattaagtgaaaaaatcaaggttttcgctacaattttcccaaaagatgatttgttactgactttcaaaaagagagacccaattactggagaagtcttcgaataa

SEQ ID NO: 52 - protein, 538 aa, Saccharomyces cerevisiae
MSSVAENIIQHATHNSTLHQLAKDQPSVGVTTAFSILDTLKSMSYLKIFATLICILLVWDQVAYQIKKGSIAGPKFKFWPIIGPFLESLDPKFEEYKAKWASGPLSCVSIFHKFVVIASTRDLARKILQSSKFVKPCVVDVAVKILRPCNWVFLDGKAHTDYRKSLNGLFTKQALAQYLPSLEQIMDKYMDKFVRLSKENNYEPQVFFHEMREILCALSLNSFCGNYITEDQVRKIADDYYLVTAALELVNFPIIIPYTKTWYGKKTADMAMKIFENCAQMAKDHIAAGGKPVCVMDAWCKLMHDAKNSNDDDSRIYHREFTNKEISEAVFTFLFASQDASSSLACWLFQIVADRPDVLAKIREEQLAVRNNDMSTELNLDLIEKMKYTNMVIKETLRYRPPVLMVPYVVKKNFPVSPNYTAPKGAMLIPTLYPALHDPEVYENPDEFIPERWVEGSKASEAKKNWLVFGCGPHVCLGQTYVMITFAALLGKFALYTDFHHTVTPLSEKIKVFATIFPKDDLLLTFKKRDPITGEVFE

SEQ ID NO: 53 - DNA, 1542 bp, Glycine max
atgaggcctctgagtctcagtctgacggagctaacctcctatgtcctatgcttcatcatcctcctgcttctcctggaacagatctcctacatactaaaaaaagcttccatcccaggaccctcctttgtacttcccttcataggcaacgctatcccattggtccgagacccaaccaatttctgggacctccaatcctcttttgctaaatccaccccctcgggcttctccgccaactacatcatcggcaacttcatcgtcttcatcagagactcccatctctcccacaaaatattctccaatgtccggcctgacgcctttcacctggtgggccaccccttcggtaaaaagctcttcggccaacacaacctcatctacatgactggccaagtccacaaagatctccgccgtcggatcgcccccaactttacacctaaagccctctccacctacaccgcgctccagcagattatcatcctcaaccacctcaagtcatggctcaatcagtcccaagccccagactcccattccattcctctccgcatcctggctcgtgacatgaacctccagacatcccagaccgtcttcgtgggcccctacttgggccccaaagcccgagagcgtttcgagagggattactttctattcaacgtcggcctaatgaagctgccgtttgacttccccggcaccgcctttcgaaacgccaggctcgccgtggaccgcctcattgcagcactgggcacgtgcaccgagatgagcaaagcacggatgaaggcagggggagagccttcgtgcctcgtcgattactggatgcaggacacgctcagggaaatcgaggaggccaagctcgccggagagatgccgccgccgttctccactgacgtcgagatcggaggttatctctttgacttcctcttcgccggccaggacgcgtccacgtcgtcgctgctttgggcggtggcgctacttgactcgcacccggaggtgctcgccaaggtgagaaccgaagtcgctggaatctggtcgccggagtccgacgagcttattactgccgacatgctgagggagatgaagtatactctggcggtggcgcgtgaggtgttgaggttccggccaccggcgacgctggtgccgcacatcgcggcggagagctttccgttgacggaatcgtacacgatacccaaaggagctatcgtgtttccgtcggcgttcgagtcgtcgtttcaagggttcactgaaccggaccggtttgacccggaccggttctcggaggagagacaggaggaccaaatatttaagagaaactttctcgcgttcggggctgggccccaccagtgtgtaggtcaaaggtacgcgttgaatcatcttgttctcttcatcgccttgttcacaacgttgatcgatttcaagagggacatatccgacggctgtgatgagattgcgtacgtgcccaccatttgccccaaagacgactgcagggtatttctgtccaaacggtgcgcacggtatccttcttttccttcggtagaggacctcgtcaaatga

SEQ ID NO: 54 - protein, 513 aa, Glycine max
MRPLSLSLTELTSYVLCFIILLLLLEQISYILKKASIPGPSFVLPFIGNAIPLVRDPTNFWDLQSSFAKSTPSGFSANYIIGNFIVFIRDSHLSHKIFSNVRPDAFHLVGHPFGKKLFGQHNLIYMTGQVHKDLRRRIAPNFTPKALSTYTALQQIIILNHLKSWLNQSQAPDSHSIPLRILARDMNLQTSQTVFVGPYLGPKARERFERDYFLFNVGLMKLPFDFPGTAFRNARLAVDRLIAALGTCTEMSKARMKAGGEPSCLVDYWMQDTLREIEEAKLAGEMPPPFSTDVEIGGYLFDFLFAGQDASTSSLLWAVALLDSHPEVLAKVRTEVAGIWSPESDELITADMLREMKYTLAVAREVLRFRPPATLVPHIAAESFPLTESYTIPKGAIVFPSAFESSFQGFTEPDRFDPDRFSEERQEDQIFKRNFLAFGAGPHQCVGQRYALNHLVLFIALFTTLIDFKRDISDGCDEIAYVPTICPKDDCRVFLSKRCARYPSFPSVEDLVK

SEQ ID NO: 55 - DNA, 102 bp, Arabidopsis thaliana
atgcagaggtttttctccgccagatcgattctcggttacgccgtcaagacgcggaggaggtctttctcttctcgttcttcgtctctcctttgctcttccatg

SEQ ID NO: 56 - protein, 34 aa, Arabidopsis thaliana
MQRFFSARSILGYAVKTRRRSFSSRSSSLLCSSM

SEQ ID NO: 57 - DNA, 102 bp, Arabidopsis thaliana
atgcagaggtttttctccgccagatcgattctcggttacgccgtcaagacgcggaggaggtctttctcttctcgttcttcggaattccagctgaccaccatg

SEQ ID NO: 58 - protein, 34 aa, Arabidopsis thaliana
MQRFFSARSILGYAVKTRRRSFSSRSSEFQLTTM

SEQ ID NO: 59 - DNA, 419 bp, Spinacia oleracea
gcataaacttatcttcatagttgccactccaatttgctccttgaatctcctccacccaatacataatccactcctccatcacccacttcactactaaatcaaacttaactctgtttttctctctcctcctttcatttcttattcttccaatcatcgtactccgccatgaccaccgctgtcaccgccgctgtttctttcccctctaccaaaaccacctctctctccgcccgaagctcctccgtcatttcccctgacaaaatcagctacaaaaaggtgattcccaatttcactgtgttttttattaataatttgttattttgatgatgagatgattaatttgggtgctgcaggttcctttgtactacaggaatgtatctgcaactgggaaaatgggacccatcagggcccagatcgcctct

SEQ ID NO: 60 - protein, 59 aa, Spinacia oleracea
MTTAVTAAVSFPSTKTTSLSARSSSVISPDKISYKKVPLYYRNVSATGKMGPIRAQIAS

SEQ ID NO: 61 - DNA, 674 bp, Vicia faba
caaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagc

SEQ ID NO: 62 - DNA, 695 bp, Vicia faba
ctagactgcagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcataccattaattaag

SEQ ID NO: 63 - DNA, 1112 bp, Artificial sequence
  Synthesized
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

SEQ ID NO: 64 - DNA, 986 bp, Petroselinum crispum
ctagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcag

(19) United States
(12) Patent Application Publication
(10) Pub. No.: US 2012/0198588 A1
(43) Pub. Date: Aug. 2, 2012

(54) TRANSGENIC PLANTS WITH ALTERED REDOX MECHANISMS AND INCREASED YIELD

(75) Inventor: Bryan D. McKersie, Raleigh, NC (US)

(73) Assignee: BASF Plant Science Company GmbH, Ludwigshafen (DE)

(21) Appl. No.: 13/138,736

(22) PCT Filed: Mar. 17, 2010
(86) PCT No.: PCT/EP2010/053470

§ 371 Date: Sep. 21, 2011

Related U.S. Application Data
(60) Provisional application No. 61/162,427, filed on Mar. 23, 2009.

(51) Int. Cl. C12N 15/82; A01H 5/00; A01H 5/10; C07H 21/04

(52) U.S. Cl. 800/290; 536/23.6; 800/298

(57) ABSTRACT

Polynucleotides are disclosed which are capable of enhancing yield of a plant transformed to contain such polynucleotides. Also provided are methods of using such polynucleotides, and transgenic plants and agricultural products, including seeds, containing such polynucleotides as transgenes.

This application claims priority benefit of U.S. provisional patent application Ser. No. 61/162,427, filed Mar. 23, 2009, the entire contents of which are incorporated herein by reference.

### FIELD OF THE INVENTION

### BACKGROUND OF THE INVENTION

Population increases and climate change have brought the possibility of global food, feed, and fuel shortages into sharp focus in recent years. Agriculture consumes 70% of water used by people, at a time when rainfall in many parts of the world is declining. In addition, as land use shifts from farms to cities and suburbs, fewer hectares of arable land are available to grow agricultural crops. Agricultural biotechnology has attempted to meet humanity's growing needs through genetic modifications of plants that could increase crop yield, for example, by conferring better tolerance to abiotic stress responses or by increasing biomass.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems.

Agricultural biotechnologists have used assays in model plant systems, greenhouse studies of crop plants, and field trials in their efforts to develop transgenic plants that exhibit increased yield, either through increases in abiotic stress tolerance or through increased biomass. For example, water use efficiency (WUE), is a parameter often correlated with drought tolerance. Studies of a plant's response to desiccation, osmotic shock, and temperature extremes are also employed to determine the plant's tolerance or resistance to abiotic stresses.

An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Agricultural biotechnologists also use measurements of other parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. There is a strong genetic component to plant size and growth rate, and so for a range of diverse genotypes plant size under one environmental condition is likely to correlate with size under another. In this way, a standard environment is used to approximate the diverse and dynamic environments encountered at different locations and times by crops in the field.

Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Plants cannot move to find sources of energy or to avoid predation or stress. As a result, plants have evolved various biochemical pathways and networks to respond to their environment that maintain the supply of energy to the developing plant under diverse environmental conditions. One of the challenges to plants under these adverse conditions, such as drought, temperature extremes and exposure to heavy metals, is that some metabolic products are highly toxic. In the case of oxidative stress, these toxins include the highly reactive oxygen species (ROS) of superoxide, peroxide, hydroxyl radicals, and organic derivatives thereof. ROS, are highly reactive towards organic molecules such as unsaturated lipids, nucleic acids and proteins. ROS abstract hydrogen from these organic molecules, leading to the formation of reduced oxygen (water or a reduced organic product) and a second organic ROS, which perpetuates a chain reaction leading to the continuous destruction of cellular components until the ROS is scavenged. Scavenging of ROS involves the formation of a non-reactive end product that is not a ROS species. A number of hydrogen donors that act as ROS scavengers are known to function in plant cells, including tocopherol, ascorbate, gluthione, and thioredoxin. These diverse ROS scavengers share two common characteristics; their oxidized form is not reactive to other organic compounds, and the oxidized form can be reduced by metabolic reactions in the cell to regenerate the reduced form of the scavenger in a cyclic reaction drawing reducing equivalents directly or indirectly from NAD(P)H.

Oxidative stress occurs in plants under adverse environmental conditions when the production of ROS formed as by-products of metabolism exceeds the capacity of the plant's scavenging systems to dissipate ROS into stable end-products. To cope with oxidative stress, the plant cell must contain adequate quantities of scavengers or enzymes capable of inactivating ROS. In addition, the cell also requires an adequate supply of reducing equivalents in the form of NAD(P)H to regenerate the active form of the scavenger. If either is inadequate, the titer of ROS increases and the cell suffers oxidative damage to lipids, nucleic acids or proteins. In severe cases, this damage may lead to cell death, necrosis and loss of productivity.

Glutathione has been detected in nearly all plant cell compartments, such as the cytosol, chloroplasts, endoplasmic reticulum, vacuoles, and mitochondria. Glutathione is the major source of non-protein thiols in plant cells; it is the chemical reactivity of the thiol group that makes glutathione involved in many biochemical functions. Glutathione is water-soluble, stable and in addition to detoxifying ROS, it also protects against other stresses such as heavy metals, organic chemicals, and pathogens. The soluble enzyme, “classic” glutathione peroxidase, converts reduced monomeric glutathione (GSH) with H₂O₂ to its oxidized form, disulfide glutathione (GSSG) and H₂O. The cellular redox balance of a cell is indicative of the GSH/GSSG ratio, and has been suggested to be involved in ROS perception and signaling. A second form of glutathione peroxidase, phospholipid hydroperoxide glutathione peroxidase (PHGPx), can be membrane-associated. PHGPx is associated with diverse functions, such as signaling and cellular differentiation, and may be linked to the thioredoxin pathway. PHGPx also reduces lipid hydroperoxides esterified to membranes. Thus, PHGPx has been associated with repair of membrane lipid peroxidation.

Glutathione is also involved in glutathionylation, which modifies proteins by protecting specific cysteine residues from irreversible oxidation, thereby regulating activity of certain proteins. The enzyme isocitrate lyase is deactivated through glutathionylation. Isocitrate lyase catalyzes the formation of succinate and glyoxylate from isocitrate, part of the glyoxylate cycle, which converts two molecules of acetyl-CoA to one succinate molecule.

Glutathione can also be degraded by the action of gamma-glutamyltranspeptidase, which catalyzes the transfer of the gamma-glutamyl moiety of glutathione to an acceptor that may be an amino acid, a peptide or water. Based on homology to animal GGTs, four genes have been found in Arabidopsis: GGT1, GGT2, GGT3, and GGT4. GGT1 accounts for 80-99% of the activity, except in seeds, where GGT2 accounts for 50% activity. Knockouts of GGT2 and GGT4 show no apparent phenotype, but GGT1 knockouts had premature senescence of rosettes shortly after flowering. Knockouts of GGT3 show reduced number of siliques and reduced seed yield.

Reduction-oxidation (redox) reactions occur when atoms undergo a change in their oxidative state, by an electron-transfer reaction. Oxidation describes a gain of oxidation state by losing hydrogen or gaining oxygen. Reduction describes a loss of oxidation state by gaining hydrogen or losing oxygen. In biology, many important energy storing or releasing pathways involve redox reactions. Cellular respiration oxidizes glucose to CO₂, and reduces O₂ to water. In photosynthesis, CO₂ is reduced to sugars and H₂O is oxidized to O₂ in Photosystem II. In Photosystem I, the electron gradient reduces cofactor NAD+ to NADH. A proton gradient is produced, driving the synthesis of ATP, as what occurs in the respiratory chain, which pumps H+ out; the H+ transporting ATP synthase couples H+ uptake to ATP synthesis. In non-photosynthetic organisms such as E. coli, redox reactions can exchange electrons and utilize hydrogen as an energy source to allow anaerobic growth, which require the action of hydrogenases.

The redox state of a cell is mainly reflective of the ratio of NAD+/NADH or NADP+/NADPH. This balance is reflected in the amount of metabolites such as pyruvate and lactate. Plant growth requires a supply of carbon, ATP, NADH and NADPH. These requirements are met by glycolysis and the pentose phosphate pathway, which provides an oxidative route for regenerating NADPH as well as a non-oxidative route for producing ribose and other pentoses from the hexoses enocuountered in metabolism. Transaldolase is an enzyme in the non-oxidative pentose phosphate pathway that catalyzes the reversible transfer of a three-carbon ketol unit from sedoheptulose-7-phosphate to glyceraldehyde-3-phosphate to form erythrose-4-phosphate and fructose-6-phosphate. Transaldolase, together with transketolase, provides a link between the glycolytic and pentose phosphate pathways.

Galactose metabolism plays a part in cellular metabolism by providing glucose for fructose and mannose metabolism, nucleotide sugar metabolism, and glycolysis. The transformation of galactose into glucose-1-phosphate requires the action of three enzymes by the Leloir pathway: galactokinase, galactose-1-phosphate uridylyltransferase, and UDP-galactose 4-epimerase. Galactokinase specifically phosphorylates galactose using ATP to form galactose-1-phosphate in the first step of the pathway.

Although some genes that are involved in stress responses, water use, and/or biomass in plants have been characterized, but to date, success at developing transgenic crop plants with improved yield has been limited, and no such plants have been commercialized. There is a need, therefore, to identify additional genes that have the capacity to increase yield of crop plants.

### SUMMARY OF THE INVENTION

The present inventors have discovered that alterations to the expression of genes related to the ROS scavenging system in plants can improve plant yield. When targeted as described herein, the polynucleotides and polypeptides set forth in Table 1 are capable of improving yield of transgenic plants.

TABLE 1

| | | Polynucleotide | Amino acid |
|---|---|---|---|
| Gene Name | Organism | SEQ ID NO | SEQ ID NO |
| b0757 | Escherichia coli | 1 | 2 |
| GM59594085 | Glycine max | 3 | 4 |
| GM59708137 | G. max | 5 | 6 |
| ZMBFb0152K10 | Zea mays | 7 | 8 |
| b2464 | E. coli | 9 | 10 |
| BN43182918 | Brassica napus | 11 | 12 |
| GM48926546 | G. max | 13 | 14 |
| b2990 | E. coli | 15 | 16 |
| YER065C | Saccaromyces | 17 | 18 |
| | cerevisiae | | |
| YIR037W | S. cerevisiae | 19 | 20 |
| BN42261838 | B. napus | 21 | 22 |
| BN43722096 | B. napus | 23 | 24 |
| BN51407729 | B. napus | 25 | 26 |
| GM50585691 | G. max | 27 | 28 |
| GMsa56c07 | G. max | 29 | 30 |
| GMsp82f11 | G. max | 31 | 32 |
| GMss66f03 | G. max | 33 | 34 |
| HA03MC1446 | Helianthus anuus | 35 | 36 |
| HV03MC9784 | Hordeum vulgare | 37 | 38 |
| OS34914218 | Oryza sativa | 39 | 40 |
| ZM61990487 | Z. mays | 41 | 42 |
| ZM68466470.r01 | Z. mays | 43 | 44 |
| slr1269 | Synechocystis sp. | 45 | 46 |
| SLL1323 | Synechocystis sp. | 47 | 48 |
| Gmsb38b04 | G. max | 49 | 50 |
| YMR015C | S. cerevisiae | 51 | 52 |
| GMso65h07 | G. max | 53 | 54 |

In one embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length galactokinase polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter and an isolated polynucleotide encoding a full-length transaldolase A polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter and an isolated polynucleotide encoding a full-length hydrogenase-2 accessory polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length isocitrate lyase polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length phospholipid hydroperoxide glutathione peroxidase polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter and an isolated polynucleotide encoding a full-length gamma-glutamyltranspeptidase polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length ATP synthase subunit B′ polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length C-22 sterol desaturase polypeptide; wherein the transgenic plant demonstrates increased yield as compared to a wild type plant of the same variety which does not comprise the expression cassette.

In a further embodiment, the invention provides a seed produced by the transgenic plant of the invention, wherein the seed is true breeding for a transgene comprising the expression vectors described above. Plants derived from the seed of the invention demonstrate increased tolerance to an environmental stress, and/or increased plant growth, and/or increased yield, under normal and/or stress conditions as compared to a wild type variety of the plant.

In a still another aspect, the invention concerns products produced by or from the transgenic plants of the invention, their plant parts, or their seeds, such as a foodstuff, feedstuff, food supplement, feed supplement, fiber, cosmetic or pharmaceutical.

The invention further provides certain isolated polynucleotides identified in Table 1, and certain isolated polypeptides identified in Table 1. The invention is also embodied in a recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by the polynucleotide. Expression of the polypeptide in the plant results in increased tolerance to an environmental stress, and/or growth, and/or yield under normal and/or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of the invention, and generating a transgenic plant from the plant cell, wherein the transgenic plant comprises the polynucleotide.

### BRIEF DESCRIPTION OF THE DRAWINGS

FIG. 1 shows an alignment of the amino acid sequences of the galactokinases designated b0757 (SEQ ID NO: 2), GM59594085 (SEQ ID NO: 4), GM59708137 (SEQ ID NO: 6), and ZMBFb0152K10 (SEQ ID NO: 8). The alignment was generated using Align X of Vector NTI.

FIG. 2 shows an alignment of the amino acid sequences of the transaldolase A proteins designated b2464 (SEQ ID NO: 10), BN43182918 (SEQ ID NO: 12), and GM48926546 (SEQ ID NO: 14). The alignment was generated using Align X of Vector NTI.

FIG. 3 shows an alignment of the amino acid sequences of the phospholipid hydroperoxide glutathione peroxidases designated YIR037W (SEQ ID NO: 20), BN42261838 (SEQ ID NO: 22), BN43722096 (SEQ ID NO: 24), BN51407729 (SEQ ID NO: 26), GM50585691 (SEQ ID NO: 28), GMsa56c07 (SEQ ID NO: 30), GMsp82f11 (SEQ ID NO: 32), GMss66f03 (SEQ ID NO: 34), HA03MC1446 (SEQ ID NO: 36), HV03MC9784 (SEQ ID NO: 38), OS34914218 (SEQ ID NO: 40), ZM61990487 (SEQ ID NO: 42), and ZM68466470.r01 (SEQ ID NO: 44). The alignment was generated using Align X of Vector NTI.

FIG. 4 shows an alignment of the amino acid sequences of the ATP synthase subunit B′ proteins designated SLL1323 (SEQ ID NO: 48) and Gmsb38b04 (SEQ ID NO: 50). The alignment was generated using Align X of Vector NTI.

FIG. 5 shows an alignment of the amino acid sequences of the C-22 sterol desaturases designated YMR015C (SEQ ID NO: 52) and GMso65h07 (SEQ ID NO: 54). The alignment was generated using Align X of Vector NTI.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1 in the subcellular compartment and tissue indicated herein. The transgenic plant of the invention demonstrates an improved yield as compared to a wild type variety of the plant. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the nucleotides and polypeptides of Table 1, as compared with the bu/acre yield from untreated soybeans or corn cultivated under the same conditions, is an improved yield in accordance with the invention.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide of the invention. The term “wild type” as used herein refers to a plant cell, seed, plant component, plant tissue, plant organ, or whole plant that has not been genetically modified with an isolated polynucleotide in accordance with the invention.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. As used herein, the term “drought” refers to an environmental condition where the amount of water available to support plant growth or development is less than optimal. As used herein, the term “fresh weight” refers to everything in the plant including water. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following diclotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, particularly the genus Brassica, which includes plant such as oilseed rape, beet, cabbage, cauliflower and broccoli); and A. thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, and the like. Especially preferred are Arabidopsis thaliana, Nicotiana tabacum, rice, oilseed rape, canola, soybean, corn (maize), cotton, and wheat.

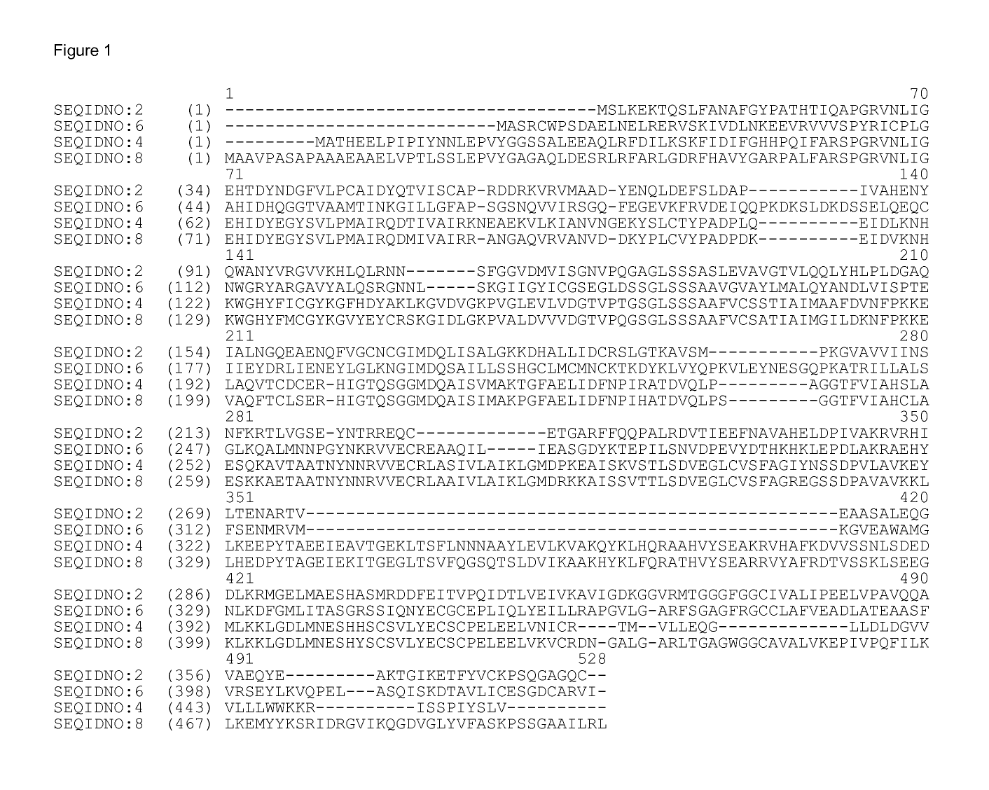

In one embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length galactokinase polypeptide; wherein the transgenic plant demonstrates increased yield as compared to a wild type plant of the same variety which does not comprise the expression cassette. As demonstrated in Example 2 below, transgenic Arabidopsis plants containing the E. coli gene b0757 (SEQ ID NO: 1) targeted to the chloroplast demonstrate increased yield as compared to control Arabidopsis plants. The b0757 gene encodes galactokinase and is characterized, in part, by the presence of the signature sequences GHMP_kinases_C (Pfam: PF08544) and GHMP_kinases_N (PF00288). Such signature sequences are exemplified in the galactokinase proteins set forth in FIG. 1.

The transgenic plant of this embodiment may comprise any polynucleotide encoding a galactokinase polypeptide. Preferably, the transgenic plant of this embodiment comprises a polynucleotide encoding a full-length polypeptide having galactokinase activity, wherein the polypeptide comprises at least one signature sequence selected from both a GHMP_kinases_C and a GHMP_kinases_N signature sequence, wherein the GHMP_kinases_C signature sequence is selected from the group consisting of amino acids 278 to 362 of SEQ ID NO: 2; amino acids 378 to 426 of SEQ ID NO: 4; amino acids 326 to 404 of SEQ ID NO: 6; and amino acids 391 to 473 of SEQ ID NO: 8; and wherein the GHMP_kinases_N signature sequence is selected from the group consisting of amino acids 114 to 182 of SEQ ID NO: 2; amino acids 152 to 219 of SEQ ID NO: 4; amino acids 138 to 205 of SEQ ID NO: 6; and amino acids 159 to 226 of SEQ ID NO: 8. Preferably the polypeptide comprises both a GHMP_kinases_C signature sequence and a GHMP_kinases_N signature sequence. Most preferably, the transgenic plant of this embodiment comprises a polynucleotide encoding a galactokinase polypeptide having a sequence selected from the group consisting of amino acids 1 to 382 of SEQ ID NO: 2; amino acids 1 to 460 of SEQ ID NO: 4; amino acids 1 to 431 of SEQ ID NO: 6; and amino acids 1 to 504 of SEQ ID NO: 8.

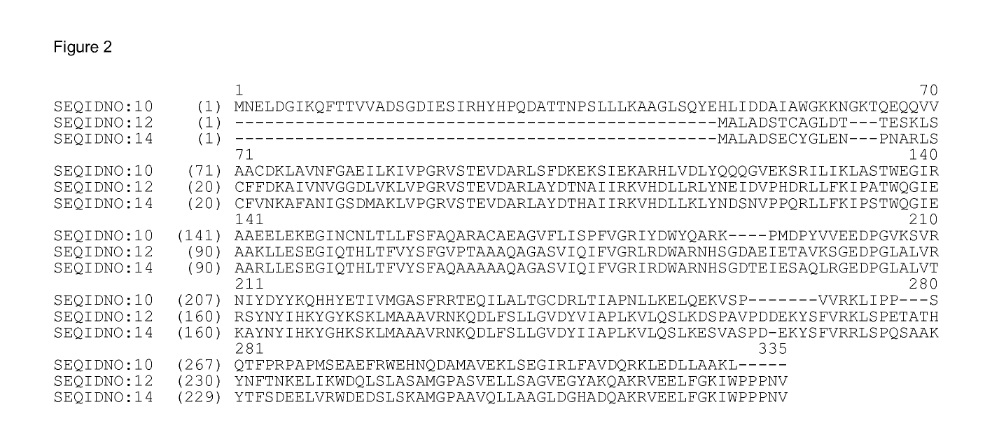

In another embodiment, the invention provides a transgenic plant transformed with an expression cassette comprising, in operative association, an isolated polynucleotide encoding a promoter; and an isolated polynucleotide encoding a full-length transaldolase A polypeptide; wherein the transgenic plant demonstrates increased yield as compared to a wild type plant of the same variety which does not comprise the expression cassette. As demonstrated in Example 2 below, transgenic Arabidopsis plants containing the E. coli gene b2464 (SEQ ID NO: 9), which encodes a transadolase A polypeptide, and the transgenic plants of this embodiment demonstrate increased yield as compared to control Arabidopsis plants. Transaldolase A polypeptides are characterized, in part, by the presence of a Transaldolase (PF00923) signature sequence. Such signature sequences are exemplified in the transaldolase A proteins set forth in FIG. 2.

The transgenic plant of this embodiment may comprise any polynucleotide encoding a transaldolase A protein. Preferably, the transgenic plant of this embodiment comprises a polynucleotide encoding a full-length polypeptide having transaldolase A activity, wherein the polypeptide comprises a Transaldolase signature sequence selected from the group consisting of amino acids 12 to 312 of SEQ ID NO: 10; amino acids 1 to 275 of SEQ ID NO: 12; and amino acids 1 to 277 of SEQ ID NO: 14. Most preferably, the transgenic plant of this embodiment comprises a polynucleotide encoding a transaldolase A polypeptide having a sequence selected from the group consisting of amino acids 1 to 316 of SEQ ID NO: 10; amino acids 1 to 284 of SEQ ID NO: 12; and amino acids 1 to 283 of SEQ ID NO: 14.

In another embodiment, the invention provides a transgenic plant transformed with an expression cassette comprising, in operative association, an isolated polynucleotide encoding a promoter; and an isolated polynucleotide encoding a full-length hydrogenase-2 accessory polypeptide; wherein the transgenic plant demonstrates increased yield as compared to a wild type plant of the same variety which does not comprise the expression cassette. As demonstrated in Example 2 below, transgenic Arabidopsis plants containing the E. coli gene b2990 (SEQ ID NO: 15) demonstrate increased yield as compared to control Arabidopsis plants. The b2990 gene encodes a hydrogenase-2 accessory protein. In E. coli under anaerobic conditions, this protein is a chaperone-like protein which is required for the generation of active hydrogenase 2, which is an uptake [NiFe] hydrogenase that, along with hydrogenase 1, couples H₂ oxidation to fumarate reduction. Hydrogenase-2 accessory proteins are characterized, in part, by the presence of a HupF_HypC (PF01455) signature sequence.

The transgenic plant of this embodiment may comprise any polynucleotide encoding a hydrogenase-2 accessory protein. Preferably, the transgenic plant of this embodiment comprises a polynucleotide encoding a full-length polypeptide having hydrogenase assembly chaperone activity, wherein the polypeptide comprises a HupF_HypC signature sequence comprising amino acids 1 to 79 of SEQ ID NO: 16. Most preferably, the transgenic plant of this embodiment comprises a polynucleotide encoding a hydrogenase-2 accessory protein having a sequence comprising amino acids 1 to 82 of SEQ ID NO: 16.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length isocitrate lyase polypeptide; wherein the transgenic plant demonstrates increased yield as compared to a wild type plant of the same variety which does not comprise the expression cassette. As demonstrated in Example 2 below, transgenic Arabidopsis plants containing the S. cerevisiae gene YER065C (SEQ ID NO: 17), which encodes isocitrate lyase, targeted to the mitochondria demonstrate increased yield as compared to control Arabidopsis plants. Isocitrate lyases are characterized, in part, by the presence of an ICL (PF00463) signature sequence.

The transgenic plant of this embodiment may comprise any polynucleotide encoding an isocitrate lyase. Preferably, the transgenic plant of this embodiment comprises a polynucleotide encoding a full-length polypeptide having isocitrate lyase activity, wherein the polypeptide comprises an ICL signature sequence comprising amino acids 22 to 550 of SEQ ID NO: 18. Most preferably, the transgenic plant of this embodiment comprises a polynucleotide encoding an isocitrate lyase having a sequence comprising amino acids 1 to 557 of SEQ ID NO: 18.

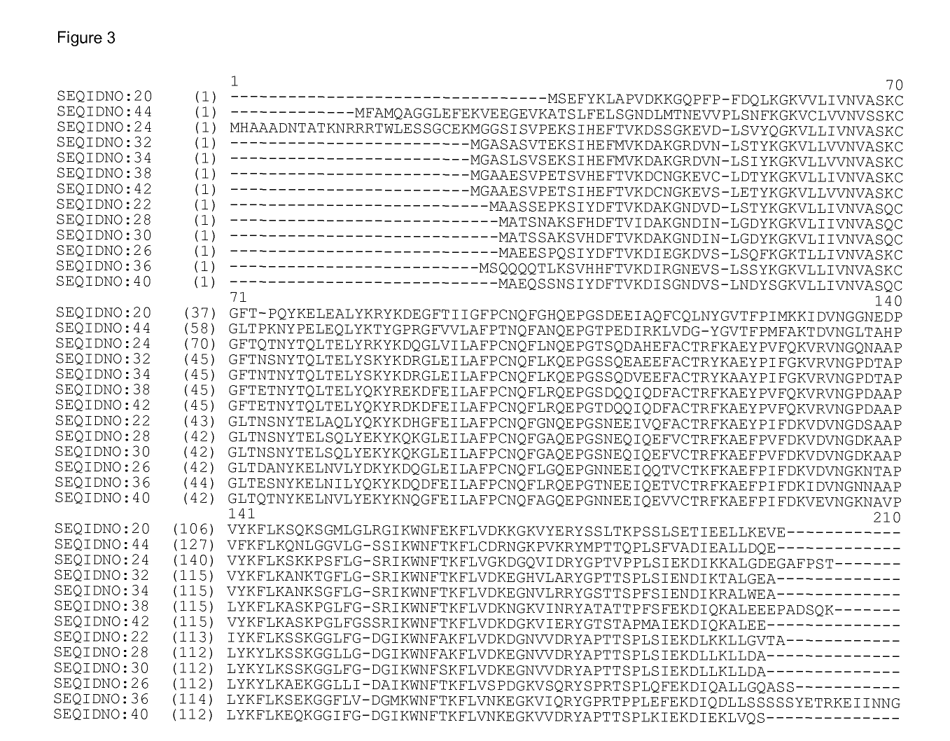

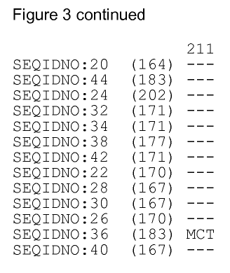

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length phospholipid hydroperoxide glutathione peroxidase polypeptide; wherein the transgenic plant demonstrates increased yield as compared to a wild type plant of the same variety which does not comprise the expression cassette. As demonstrated in Example 2 below, transgenic Arabidopsis plants containing the S. cerevisiae gene YIR037W (SEQ ID NO: 19) targeted to the chloroplast demonstrate increased yield as compared to control Arabidopsis plants. The YIR037W gene encodes encodes a phospholipid hydroperoxide glutathione peroxidase protein, which functions as a sensor for intracellular hyperoxide levels, and a transducer of the redox signal to the transcription factor Yap1, which regulates hyperoxide levels in S. cerevisiae. Phospholipid hydroperoxide glutathione peroxidases are characterized, in part, by the presence of a GSHPx (PF00255) signature sequence representative of the glutathione peroxidase family of genes. Such signature sequences are exemplified in the phospholipid hydroperoxide glutathione peroxidases set forth in FIG. 3.

The transgenic plant of this embodiment may comprise any polynucleotide encoding a phospholipid hydroperoxide glutathione peroxidase. Preferably, the transgenic plant of this embodiment comprises a polynucleotide encoding a full-length polypeptide having phospholipid hydroperoxide glutathione peroxidase activity, wherein the polypeptide comprises a GSHPx signature sequence selected from the group consisting of amino acids 4 to 111 of SEQ ID NO: 20; amino acids 10 to 118 of SEQ ID NO: 22; amino acids 37 to 145 of SEQ ID NO: 24; amino acids 9 to 117 of SEQ ID NO: 26; amino acids 9 to 117 of SEQ ID NO: 28; amino acids 9 to 117 of SEQ ID NO: 30; amino acids 12 to 120 of SEQ ID NO: 32; amino acids 12 to 120 of SEQ ID NO: 34; amino acids 11 to 119 of SEQ ID NO: 36; amino acids 12 to 120 of SEQ ID NO: 38; amino acids 9 to 117 of SEQ ID NO: 40; amino acids 12 to 120 of SEQ ID NO: 42; and amino acids 24 to 132 of SEQ ID NO: 44. Most preferably, the transgenic plant of this embodiment comprises a polynucleotide encoding a phospholipid hydroperoxide glutathione peroxidase having a sequence selected from the group consisting of amino acids 1 to 163 of SEQ ID NO: 20; amino acids 1 to 169 of SEQ ID NO: 22; amino acids 1 to 201 of SEQ ID NO: 24; amino acids 1 to 169 of SEQ ID NO: 26; amino acids 1 to 166 of SEQ ID NO: 28; amino acids 1 to 166 of SEQ ID NO: 30; amino acids 1 to 170 of SEQ ID NO: 32; amino acids 1 to 170 of SEQ ID NO: 34; amino acids 1 to 185 of SEQ ID NO: 36; amino acids 1 to 176 of SEQ ID NO: 38; amino acids 1 to 166 of SEQ ID NO: 40; amino acids 1 to 170 of SEQ ID NO: 42; and amino acids 1 to 182 of SEQ ID NO: 44.

In another embodiment, the invention provides a transgenic plant transformed with an expression cassette comprising, in operative association, an isolated polynucleotide encoding a promoter; and an isolated polynucleotide encoding a full-length gamma-glutamyltranspeptidase polypeptide; wherein the transgenic plant demonstrates increased yield as compared to a wild type plant of the same variety which does not comprise the expression cassette. Optionally, the expression cassette further comprises an isolated polynucleotide encoding a chloroplast transit peptides in operative association with the isolated polynucleotide encoding a promoter and the isolated polynucleotide encoding a full-length gamma-glutamyltranspeptidase polypeptide. As demonstrated in Example 2 below, transgenic Arabidopsis plants containing the Synechocystis sp. gene slr1269 (SEQ ID NO: 45), which encodes a gamma-glutamyltranspeptidase polypeptide, demonstrate increased yield as compared to control Arabidopsis plants. Gamma-glutamyltranspeptidases are characterized, in part, by the presence of a G_glu_transpept (PF01019) signature sequence.

The transgenic plant of this embodiment may comprise any polynucleotide encoding a gamma-glutamyltranspeptidase. Preferably, the transgenic plant of this embodiment comprises a polynucleotide encoding a full-length polypeptide having gamma-glutamyltranspeptidase activity, wherein the polypeptide comprises a G_glu_transpept signature sequence comprising amino acids 21 to 511 of SEQ ID NO: 46. Most preferably, the transgenic plant of this embodiment comprises a polynucleotide encoding a gamma-glutamyltranspeptidase having a sequence comprising amino acids 1 to 518 of SEQ ID NO: 46.

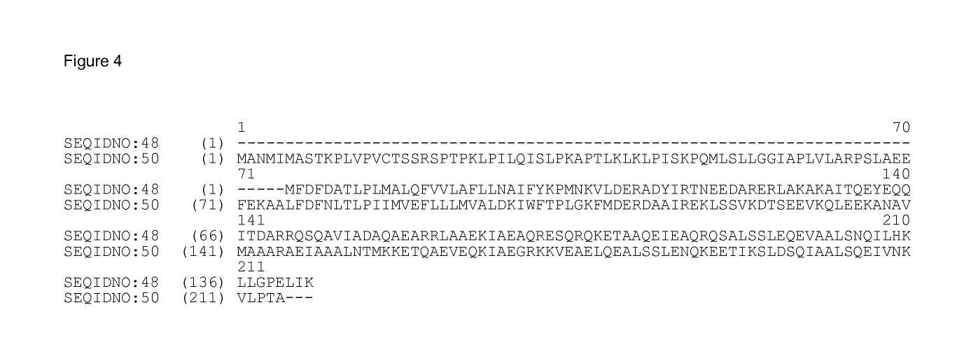

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length ATP synthase subunit B′ polypeptide; wherein the transgenic plant demonstrates increased yield as compared to a wild type plant of the same variety which does not comprise the expression cassette. As demonstrated in Example 2 below, transgenic Arabidopsis plants containing the Synechocystis sp. gene SLL1323 (SEQ ID NO: 47) targeted to the mitochondria demonstrate increased yield as compared to control Arabidopsis plants. The SLL1323 gene encodes an ATP synthase subunit B′ protein. Subunits B and B′ are from the F0 complex in F-ATPases found in chloroplasts and in bacterial plasma membranes and form part of the peripheral stalk that links the F1 and F0 complexes together. ATP synthase subunit B′ proteins are characterized, in part, by the presence of an ATP-synt_B (PF00430) signature sequence representative of the ATP synthase B/ B′ CF(0) family of genes. Such signature sequences are exemplified in the ATP synthase subunit B′ proteins set forth in FIG. 4.

The transgenic plant of this embodiment may comprise any polynucleotide encoding an ATP synthase subunit B′ protein. Preferably, the transgenic plant of this embodiment comprises a polynucleotide encoding a full-length polypeptide having ATP synthase subunit B′ activity, wherein the polypeptide comprises a ATP-synt_B signature sequence selected from the group consisting of amino acids 7 to 138 of SEQ ID NO: 48 and amino acids 82 to 213 of SEQ ID NO: 50. Most preferably, the transgenic plant of this embodiment comprises a polynucleotide encoding a ATP synthase subunit B′ protein having a sequence comprising amino acids 1 to 143 of SEQ ID NO: 48 and amino acids 1 to 215 of SEQ ID NO: 50.

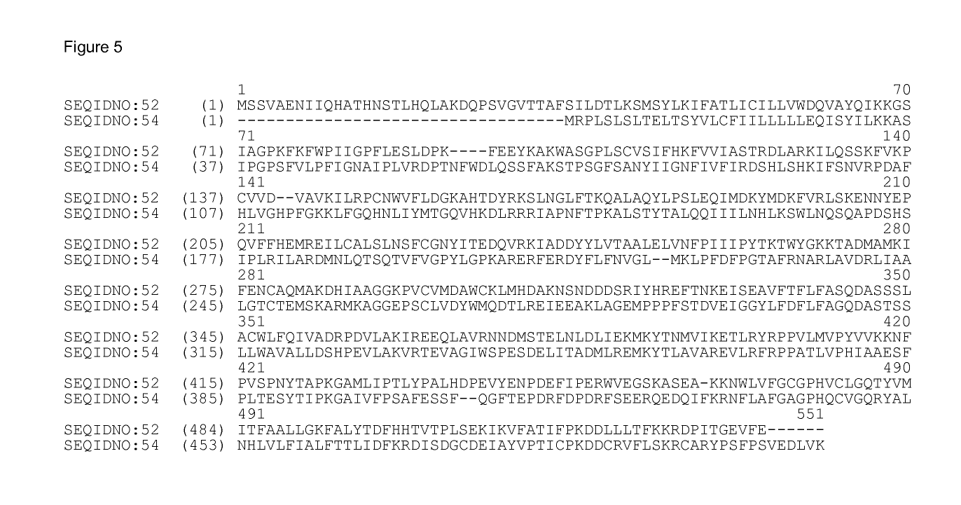

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length C-22 sterol desaturase polypeptide; wherein the transgenic plant demonstrates increased yield as compared to a wild type plant of the same variety which does not comprise the expression cassette. Gene YMR015C (SEQ ID NO: 51) encodes C-22 sterol desaturase, which is a cytochrome P450 enzyme (ERG5) that, in yeast, catalyzes the formation of the C-22(23) double bond in the sterol side chain in ergosterol biosynthesis. C-22 sterol desaturase enzymes are characterized, in part, by the presence of a K-helix motif (xExxR), a PERF consensus sequence (PxRx) and an FGRCG motif surrounding the protoporphyrin IX heme cysteine ligand near the C-terminus. Such conserved motifs are exemplified in the C-22 sterol desaturase polypeptides set forth in FIG. 5.

The transgenic plant of this embodiment may comprise any polynucleotide encoding a C-22 sterol desaturase. Preferably, the transgenic plant of this embodiment comprises a polynucleotide encoding a full-length polypeptide having C-22 sterol desaturase activity, wherein the polypeptide comprises a domain comprising a K-helix motif, a PERF motif and a FGRCG motif, wherein the K-helix motif has a sequence selected from the group consisting of amino acids 395 to 398 of SEQ ID NO: 52 and amino acids 365 to 368 of SEQ ID NO: 54; the PERF motif has a sequence selected from the group consisting of amino acids 450 to 453 of SEQ ID NO: 52 and amino acids 418 to 421 of SEQ ID NO: 54; and the FGRCG motif has a sequence selected from the group consisting of amino acids 469 to 478 of SEQ ID NO: 52 and amino acids 438 to 447 of SEQ ID NO: 54. More preferably, the polynucleotide encodes a full-length polypeptide having C-22 sterol desaturase activity, wherein the polypeptide comprises a domain selected from the group consisting of amino acids 61 to 529 of SEQ ID NO: 52 and amino acids 27 to 498 of SEQ ID NO: 54. Most preferably, the transgenic plant of this embodiment comprises a polynucleotide encoding a C-22 sterol desaturase comprising amino acids 1 to 538 of SEQ ID NO: 52 and amino acids 1 to 513 of SEQ ID NO: 54.

The invention further provides a seed which is true breeding for the expression cassettes (also referred to herein as “transgenes”) described herein, wherein transgenic plants grown from said seed demonstrate increased yield as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

The invention also provides an isolated polynucleotide which has a sequence selected from the group consisting of SEQ ID NO: 3; SEQ ID NO: 5; SEQ ID NO: 7; SEQ ID NO: 11; SEQ ID NO: 13; SEQ ID NO: 21; SEQ ID NO: 23; SEQ ID NO: 25; SEQ ID NO: 27; SEQ ID NO: 29; SEQ ID NO: 31; SEQ ID NO: 33; SEQ ID NO: 35; SEQ ID NO: 37; SEQ ID NO: 39; SEQ ID NO: 41; SEQ ID NO: 43; SEQ ID NO: 49; and SEQ ID NO: 53. Also encompassed by the isolated polynucleotide of the invention is an isolated polynucleotide encoding a polypeptide having an amino acid sequence selected from the group consisting of SEQ ID NO: 4; SEQ ID NO: 6; SEQ ID NO: 8; SEQ ID NO: 12; SEQ ID NO: 14; SEQ ID NO: 22; SEQ ID NO: 24; SEQ ID NO: 26; SEQ ID NO: 28; SEQ ID NO: 30; SEQ ID NO: 32; SEQ ID NO: 34; SEQ ID NO: 36; SEQ ID NO: 38; SEQ ID NO: 40; SEQ ID NO: 42; SEQ ID NO: 44; SEQ ID NO: 50; and SEQ ID NO: 54. A polynucleotide of the invention can be isolated using standard molecular biology techniques and the sequence information provided herein, for example, using an automated DNA synthesizer.

The isolated polynucleotides of the invention include homologs of the polynucleotides of Table 1. “Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from one of the nucleotide sequences shown in Table 1 due to degeneracy of the genetic code and thus encode the same polypeptide.

To determine the percent sequence identity of two amino acid sequences (e.g., one of the polypeptide sequences of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the isolated amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence identified in Table 1. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in Table 1.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI 9.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of the polypeptides listed in Table 1 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10× Denhart' s solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

The isolated polynucleotides employed in the invention may be optimized, that is, genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

The invention further provides a recombinant expression vector which comprises an expression cassette selected from the group consisting of a) an expression cassette comprising, in operative association, an isolated polynucleotide encoding a promoter; an isolated polynucleotide encoding a chloroplast transit peptide; and an isolated polynucleotide encoding a full-length galactokinase polypeptide; b) an expression cassette comprising, in operative association, an isolated polynucleotide encoding a promoter; and an isolated polynucleotide encoding a full-length transaldolase A polypeptide; c) an expression cassette comprising, in operative association, an isolated polynucleotide encoding a promoter; and an isolated polynucleotide encoding a full-length hydrogenase-2 accessory polypeptide; d) an expression cassette comprising, in operative association, an isolated polynucleotide encoding a promoter; an isolated polynucleotide encoding a mitochondrial transit peptide; and an isolated polynucleotide encoding a full-length isocitrate lyase polypeptide; e) an expression cassette comprising, in operative association, an isolated polynucleotide encoding a promoter; an isolated polynucleotide encoding a chloroplast transit peptide; and an isolated polynucleotide encoding a full-length phospholipid hydroperoxide glutathione peroxidase polypeptide; f) an expression cassette comprising, in operative association, an isolated polynucleotide encoding a promoter; and an isolated polynucleotide encoding a full-length gamma-glutamyltranspeptidase polypeptide; g) an expression cassette comprising, in operative association, an isolated polynucleotide encoding a promoter; an isolated polynucleotide encoding a mitochondrial transit peptide; and an isolated polynucleotide encoding a full-length ATP synthase subunit B′ polypeptide; and h) an expression cassette comprising, in operative association, an isolated polynucleotide encoding a promoter; an isolated polynucleotide encoding a chloroplast transit peptide; and an isolated polynucleotide encoding a full-length C-22 sterol desaturase polypeptide.

In another embodiment, the recombinant expression vector of the invention comprises an isolated polynucleotide having a sequence selected from the group consisting of SEQ ID NO: 3; SEQ ID NO: 5; SEQ ID NO: 7; SEQ ID NO: 11; SEQ ID NO: 13; SEQ ID NO: 21; SEQ ID NO: 23; SEQ ID NO: 25; SEQ ID NO: 27; SEQ ID NO: 29; SEQ ID NO: 31; SEQ ID NO: 33; SEQ ID NO: 35; SEQ ID NO: 37; SEQ ID NO: 39; SEQ ID NO: 41; SEQ ID NO: 43; SEQ ID NO: 49; and SEQ ID NO: 53. In addition, the recombinant expression vector of the invention comprises an isolated polynucleotide encoding a polypeptide having an amino acid sequence selected from the group consisting of SEQ ID NO: 4; SEQ ID NO: 6; SEQ ID NO: 8; SEQ ID NO: 12; SEQ ID NO: 14; SEQ ID NO: 22; SEQ ID NO: 24; SEQ ID NO: 26; SEQ ID NO: 28; SEQ ID NO: 30; SEQ ID NO: 32; SEQ ID NO: 34; SEQ ID NO: 36; SEQ ID NO: 38; SEQ ID NO: 40; SEQ ID NO: 42; SEQ ID NO: 44; SEQ ID NO: 50; and SEQ ID NO: 54.

The recombinant expression vector of the invention also include one or more regulatory sequences, selected on the basis of the host cells to be used for expression, which is in operative association with the isolated polynucleotide to be expressed. As used herein with respect to a recombinant expression vector, “in operative association” or “operatively linked” means that the polynucleotide of interest is linked to the regulatory sequence(s) in a manner which allows for expression of the polynucleotide when the vector is introduced into the host cell (e.g., in a bacterial or plant host cell). The term “regulatory sequence” is intended to include promoters, enhancers, and other expression control elements (e.g., polyadenylation signals).

As set forth above, certain embodiments of the invention employ promoters that are capable of enhancing gene expression in leaves. In some embodiments, the promoter is a leaf-specific promoter. Any leaf-specific promoter may be employed in these embodiments of the invention. Many such promoters are known, for example, the USP promoter from Vicia faba (Baeumlein et al. (1991) Mol. Gen. Genet. 225, 459-67), promoters of light-inducible genes such as ribulose-1.5-bisphosphate carboxylase (rbcS promoters), promoters of genes encoding chlorophyll a/b-binding proteins (Cab), Rubisco activase, B-subunit of chloroplast glyceraldehyde 3-phosphate dehydrogenase from A. thaliana, (Kwon et al. (1994) Plant Physiol. 105, 357-67) and other leaf-specific promoters such as those identified in Aleman, I. (2001) Isolation and characterization of leaf-specific promoters from alfalfa (Medicago sativa), Masters thesis, New Mexico State University, Los Cruces, N. Mex.

In other embodiments of the invention, a root- or shoot-specific promoter is employed. For example, the Super promoter provides high level expression in both root and shoots (Ni et al. (1995) Plant J. 7: 661-676). Other root-specific promoters include, without limitation, the TobRB7 promoter (Yamamoto et al. (1991) Plant Cell 3, 371-382), the roID promoter (Leach et al. (1991) Plant Science 79, 69-76); CaMV 35S Domain A (Benfey et al. (1989) Science 244, 174-181), and the like.

In other embodiments, a constitutive promoter is employed. Constitutive promoters are active under most conditions. Examples of constitutive promoters suitable for use in these embodiments include the parsley ubiquitin promoter described in WO2003/102198; the CaMV 19S and 35S promoters, the sX CaMV 35S promoter, the Sep1 promoter, the rice actin promoter, the Arabidopsis actin promoter, the maize ubiquitin promoter, pEmu, the figwort mosaic virus 35S promoter, the Smas promoter, the super promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In accordance with the invention, a chloroplast transit sequence refers to a nucleotide sequence that encodes a chloroplast transit peptide. Examples of a chloroplast transit peptide include the group consisting of chlorophyll a/b binding protein transit peptide, small subunit of ribulose bisphosphate carboxylase transit peptide, EPSPS transit peptide, and dihydrodipocolinic acid synthase transit peptide. As defined herein, a mitochondrial transit sequence refers to a nucleotide sequence that encodes a mitochondrial presequence and directs the protein to mitochondria. Examples of mitochondrial presequences include groups consisting of ATPase subunits, ATP synthase subunits, Rieske-FeS protein, Hsp60, malate dehydrogenase, citrate synthase, aconitase, isocitrate dehydrogenase, pyruvate dehydrogenase, malic enzyme, glycine decarboxylase, serine hydroxymethyl transferase and superoxide dismutase.

Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example any of the techniques described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Canola may be transformed, for example, using methods such as those disclosed in U.S. Pat. Nos. 5,188,958; 5,463,174; 5,750,871; EP1566443; WO02/00900; and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

The invention is also embodied in a method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression cassette described above, (b) regenerating a transgenic plant from the transformed plant cell; and selecting higher-yielding plants from the regenerated plant sells. The plant cell may be, but is not limited to, a protoplast, gamete producing cell, and a cell that regenerates into a whole plant. As used herein, the term “transgenic” refers to any plant, plant cell, callus, plant tissue, or plant part, that contains the expression cassette described above. In accordance with the invention, the expression cassette is stably integrated into a chromosome or stable extra-chromosomal element, so that it is passed on to successive generations.

The effect of the genetic modification on plant growth and/or yield and/or stress tolerance can be assessed by growing the modified plant under normal and/or less than suitable conditions and then analyzing the growth characteristics and/or metabolism of the plant. Such analytical techniques are well known to one skilled in the art, and include measurements of dry weight, wet weight, seed weight, seed number, polypeptide synthesis, carbohydrate synthesis, lipid synthesis, evapotranspiration rates, general plant and/or crop yield, flowering, reproduction, seed setting, root growth, respiration rates, photosynthesis rates, metabolite composition, and the like.

The invention is further illustrated by the following examples, which are not to be construed in any way as imposing limitations upon the scope thereof.

### EXAMPLE 1

### Characterization of Genes

Lead genes b0757 (SEQ ID NO: 1), b2464 (SEQ ID NO: 9), b2990 (SEQ ID NO: 15), SLL1323 (SEQ ID NO: 47), slr1269 (SEQ ID NO: 45), YER065C (SEQ ID NO: 17), YIR037W (SEQ ID NO: 19), and YMR015C (SEQ ID NO: 51) were cloned using standard recombinant techniques. The functionality of each lead gene was predicted by comparing the amino acid sequence encoded by the gene with other genes of known functionality. Homolog cDNAs were isolated from proprietary libraries of the respective species using known methods. Sequences were processed and annotated using bioinformatics analyses.

The b0757 gene (SEQ ID NO: 1) from E. coli encodes a galactokinase. The full-length amino acid sequence of b0757 (SEQ ID NO: 2) was blasted against a proprietary database of cDNAs at an e value of e⁻¹⁰ (Altschul et al., supra). Two homologs from soybean and one homolog from maize were identified. The amino acid relatedness of these sequences is indicated in the alignments shown in FIG. 1.

The b2464 gene (SEQ ID NO: 9) from E. coli encodes transaldolase A. The full-length amino acid sequence of b2464 (SEQ ID NO: 10) was blasted against a proprietary database of cDNAs at an e value of e⁻¹⁰ (Altschul et al., supra). One homolog from canola and one homolog from soybean were identified. The amino acid relatedness of these sequences is indicated in the alignments shown in FIG. 2.

The YIR037W gene (SEQ ID NO: 19) from S. cerevisiae encodes phospholipid hydroperoxide glutathione peroxidase. The full-length amino acid sequence of YIR037W (SEQ ID NO: 20) was blasted against a proprietary database of cDNAs at an e value of e⁻¹⁰ (Altschul et al., supra). Three homologs from canola, four homologs from soybean, one homolog from sunflower, one homolog from barley, one homolog from rice, and two homologs from maize were identified. The amino acid relatedness of these sequences is indicated in the alignments shown in FIG. 3.

The SLL1323 gene (SEQ ID NO: 47) from Synechocystis sp. encodes ATP synthase subunit B′. The full-length amino acid sequence of SLL1323 (SEQ ID NO: 48) was blasted against a proprietary database of cDNAs at an e value of e¹⁰ (Altschul et al., supra). One homolog from soybean was identified. The amino acid relatedness of these sequences is indicated in the alignments shown in FIG. 4.

The YMR015C gene (SEQ ID NO: 51) from S. cerevisiae encodes C-22 sterol desaturase. The full-length amino acid sequence of YMR015C SEQ ID NO: 52) was blasted against a proprietary database of cDNAs at an e value of e⁻¹⁰ (Altschul et al., supra). One homolog from soybean was identified. The amino acid relatedness of these sequences is indicated in the alignments shown in FIG. 5.

### EXAMPLE 2

### Overexpression of Lead Genes in Plants

The polynucleotides of Table 1 were ligated into an expression cassette using known methods. Three different promoters were used to control expression of the transgenes in Arabidopsis: the USP promoter (“USP”) from Vicia faba (SEQ ID NO: 61 or SEQ ID NO: 62); the super promoter (“Super”; SEQ ID NO: 63); and the parsley ubiquitin promoter (“PCUbi”; SEQ ID NO: 64). For targeted expression, a mitochondrial transit peptide (SEQ ID NO: 56 or SEQ ID NO: 58; designated “Mito” in Tables 2-9) or a chloroplast transit peptide (SEQ ID NO: 60; designated “Plastid” in Tables 2-10) was used.

The Arabidopsis ecotype C24 was transformed with constructs containing the lead genes described in Example 1 using known methods. Seeds from T2 transformed plants were pooled on the basis of the promoter driving the expression, gene source species and type of targeting (chloroplast, mitochondrial, or no targeting). The seed pools were used in the primary screens for biomass under well watered and water limited growth conditions. Hits from pools in the primary screen were selected, molecular analysis performed and seed collected. The collected seeds were then used for analysis in secondary screens where a larger number of individuals for each transgenic event were analyzed. If plants from a construct were identified in the secondary screen as having increased biomass compared to the controls, it passed to the tertiary screen. In this screen, over 100 plants from all transgenic events for that construct were measured under well watered and drought growth conditions. The data from the transgenic plants were compared to wild type Arabidopsis plants or to plants grown from a pool of randomly selected transgenic Arabidopsis seeds using standard statistical procedures.

Plants that were grown under well watered conditions were watered to soil saturation twice a week. Images of the transgenic plants were taken at 17 and 21 days using a commercial imaging system. Alternatively, plants were grown under water limited growth conditions by watering to soil saturation infrequently which allowed the soil to dry between watering treatments. In these experiments, water was given on days 0, 8, and 19 after sowing. Images of the transgenic plants were taken at 20 and 27 days using a commercial imaging system.

Image analysis software was used to compare the images of the transgenic and control plants grown in the same experiment. The images were used to determine the relative size or biomass of the plants as pixels and the color of the plants as the ratio of dark green to total area. The latter ratio, termed the health index, was a measure of the relative amount of chlorophyll in the leaves and therefore the relative amount of leaf senescence or yellowing and was recorded at day 27 only. Variation exists among transgenic plants that contain the various lead genes, due to different sites of DNA insertion and other factors that impact the level or pattern of gene expression. To show this effect the data tables indicate the number of plants that were positive and negative for the trait.

Tables 2 to 9 show the comparison of measurements of the Arabidopsis plants. “CD” indicates that the plants were grown under cycling drought conditions; “WW” indicates well-watered conditions. A number after an abbreviation indicates multiple independent experiments under the same conditions. Percent change indicates the measurement of the transgenic relative to the control plants as a percentage of the control non-transgenic plants; p value is the statistical significance of the difference between transgenic and control plants based on a T-test comparison of all independent events where NS indicates not significant at the 5% level of probabilty; No. of events indicates the total number of independent transgenic events tested in the experiment; No. of positive events indicates the total number of independent transgenic events that were larger than the control in the experiment; No. of negative events indicates the total number of independent transgenic events that were smaller than the control in the experiment.

### A. Galactokinase

The galactokinase gene b0757 (SEQ ID NO: 1) was expressed in Arabidopsis under control of the Super promoter with targeting to the chloroplast. Table 2 sets forth biomass and health index data obtained from the Arabidopsis plants transformed with these constructs and tested under well-watered and cycling drought conditions.

TABLE 2

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW | b0757 | Super | Plastid | Biomass at | −1.5 | NS | 7 | 3 | 4 |
| | | | | Day 17 | | | | | |
| WW | b0757 | Super | Plastid | Biomass at | 0.1 | NS | 7 | 3 | 4 |
| | | | | Day 21 | | | | | |
| WW | b0757 | Super | Plastid | Health | −2.0 | NS | 7 | 3 | 4 |
| | | | | Index | | | | | |
| CD | b0757 | Super | Plastid | Biomass at | 8.4 | 0.014 | 4 | 4 | 0 |
| | | | | Day 20 | | | | | |
| CD | b0757 | Super | Plastid | Biomass at | 8.0 | 0.026 | 4 | 4 | 0 |
| | | | | Day 27 | | | | | |
| CD | b0757 | Super | Plastid | Health | −0.9 | NS | 4 | 2 | 2 |
| | | | | Index | | | | | |

Table 2 shows that Arabidopsis plants expressing the b0757 gene targeted to the chloroplast resulted in plants that were larger under water limiting conditions, but not under well-watered conditions. In these experiments, all independent transgenic events expressing the b0757 gene were larger than the controls indicating better adaptation to the stress environment.

### B. Transaldolase A

The transaldolase A gene b2464 (SEQ ID NO: 9) was expressed in Arabidopsis under control of the USP or the Super promoter with no subcellular targeting. Table 3 sets forth biomass and health index data obtained from the Arabidopsis plants transformed with these constructs and tested under well-watered and cycling drought conditions.

TABLE 3

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW | b2464 | USP | None | Biomass at | 27.4 | 0.000 | 6 | 6 | 0 |
| | | | | Day 17 | | | | | |
| WW | b2464 | USP | None | Biomass at | 14.2 | 0.000 | 6 | 6 | 0 |
| | | | | Day 21 | | | | | |
| WW | b2464 | USP | None | Health | 6.0 | NS | 6 | 4 | 2 |
| | | | | Index | | | | | |
| CD | b2464 | Super | None | Biomass at | 18.8 | 0.000 | 5 | 4 | 1 |
| | | | | Day 20 | | | | | |
| CD | b2464 | Super | None | Biomass at | 11.2 | 0.000 | 5 | 4 | 1 |
| | | | | Day 27 | | | | | |
| CD | b2464 | Super | None | Health | 2.5 | NS | 5 | 2 | 3 |
| | | | | Index | | | | | |

Table 3 shows that Arabidopsis plants expressing the b2464 gene under control of the Super promoter were larger under water limiting conditions. Variation does exist among transgenic plants that contain the b2464 gene, due to different sites of DNA insertion and other factors that impact the level or pattern of gene expression. In these experiments, the majority of independent transgenic events expressing the b2464 gene were larger than the controls indicating better adaptation to the stress environment. Additionally, expression of the b2464 gene under control of the USP promoter resulted in plants that were larger under well-water conditions. In these experiments, all transgenic events expressing the b2464 gene were larger than the controls.

### C. Hydrogenase-2 Accessory Protein

The hydrogenase-2 accessory protein gene b2990 (SEQ ID NO: 15) was expressed in Arabidopsis under control of the Super promoter with no subcellular targeting. Table 4 sets forth biomass and health index data obtained from the Arabidopsis plants transformed with these constructs and tested under well-watered and cycling drought conditions.

TABLE 4

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW | b2990 | Super | None | Biomass | 9.3 | 0.0084 | 6 | 5 | 1 |
| | | | | at Day 17 | | | | | |
| WW | b2990 | Super | None | Biomass | 11.1 | 0.0001 | 6 | 5 | 1 |
| | | | | at Day 21 | | | | | |
| WW | b2990 | Super | None | Health | −7.4 | 0.0120 | 6 | 0 | 6 |
| | | | | Index | | | | | |
| CD | b2990 | Super | None | Biomass | 19.4 | 0.0000 | 6 | 6 | 0 |
| | | | | at Day 20 | | | | | |
| CD | b2990 | Super | None | Biomass | 21.9 | 0.0000 | 6 | 6 | 0 |
| | | | | at Day 27 | | | | | |
| CD | b2990 | Super | None | Health | 1.9 | NS | 6 | 5 | 1 |
| | | | | Index | | | | | |

Table 4 shows that Arabidopsis plants expressing the b2990 gene were larger under both well-watered and water limiting conditions. Variation does exist among transgenic plants that contain the b2990 gene, due to different sites of DNA insertion and other factors that impact the level or pattern of gene expression. In these experiments, the majority of independent transgenic events expressing the b2990 gene were larger than the controls indicating better adaptation to the stress environment. Under well-watered conditions, expression of the b2990 gene resulted in plants with reduced health index; this effect was not seen under water limiting conditions.

### D. Isocitrate Lyase

The isocitrate lyase gene YER065C (SEQ ID NO: 17) was expressed in Arabidopsis under control of the USP promoter with targeting to the mitochondria. Table 5 sets forth biomass and health index data obtained from the Arabidopsis plants transformed with these constructs and tested under well-watered conditions.

TABLE 5

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW1 | YER065C | USP | Mito | Biomass at | 7.5 | 0.0136 | 7 | 5 | 2 |
| | | | | Day 17 | | | | | |
| WW1 | YER065C | USP | Mito | Biomass at | 0.9 | NS | 7 | 4 | 3 |
| | | | | Day 21 | | | | | |
| WW1 | YER065C | USP | Mito | Health | 1.3 | NS | 7 | 3 | 4 |
| | | | | Index | | | | | |
| WW2 | YER065C | USP | Mito | Biomass at | 30.6 | 0.0000 | 8 | 8 | 0 |
| | | | | Day 17 | | | | | |
| WW2 | YER065C | USP | Mito | Biomass at | 22.1 | 0.0000 | 8 | 8 | 0 |
| | | | | Day 21 | | | | | |
| WW2 | YER065C | USP | Mito | Health | 14.6 | 0.0000 | 8 | 7 | 1 |
| | | | | Index | | | | | |

Table 5 shows that Arabidopsis plants expressing the YER065C gene were larger under well-watered conditions. Variation does exist among transgenic plants that contain the YER065C gene, due to different sites of DNA insertion and other factors that impact the level or pattern of gene expression. In these experiments, the majority of the independent transgenic events expressing the YER065C gene were larger than the controls.

### E. Phospholipid Hydroperoxide Glutathione Peroxidase

The phospholipid hydroperoxide glutathione peroxidase gene YIR037W (SEQ ID NO: 19) was expressed in Arabidopsis under control of the USP or the PCUbi promoter with targeting to the chloroplast or to the mitochondria. Table 6 sets forth biomass and health index data obtained from the Arabidopsis plants transformed with these constructs and tested under well-watered or water limiting conditions.

TABLE 6

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW | YIR037W | PCUbi | Plastid | Biomass | 4.9 | NS | 6 | 4 | 2 |
| | | | | at Day 17 | | | | | |
| WW | YIR037W | PCUbi | Plastid | Biomass | −1.8 | NS | 6 | 3 | 3 |
| | | | | at Day 21 | | | | | |
| WW | YIR037W | PCUbi | Plastid | Health | 11.3 | 0.006 | 6 | 6 | 0 |
| | | | | Index | | | | | |
| WW | YIR037W | USP | Plastid | Biomass | −12.1 | 0.003 | 6 | 1 | 5 |
| | | | | at Day 17 | | | | | |
| WW | YIR037W | USP | Plastid | Biomass | −8.1 | 0.017 | 6 | 1 | 5 |
| | | | | at Day 21 | | | | | |
| WW | YIR037W | USP | Plastid | Health | −7.5 | 0.000 | 6 | 0 | 6 |
| | | | | Index | | | | | |
| CD | YIR037W | PCUbi | Plastid | Biomass | 12.2 | 0.004 | 6 | 5 | 1 |
| | | | | at Day 20 | | | | | |
| CD | YIR037W | PCUbi | Plastid | Biomass | 11.2 | 0.000 | 6 | 6 | 0 |
| | | | | at Day 27 | | | | | |
| CD | YIR037W | PCUbi | Plastid | Health | 10.2 | 0.011 | 6 | 5 | 1 |
| | | | | Index | | | | | |
| CD | YIR037W | USP | Mito | Biomass | −6.1 | NS | 6 | 2 | 4 |
| | | | | at Day 20 | | | | | |
| CD | YIR037W | USP | Mito | Biomass | −6.0 | NS | 6 | 1 | 5 |
| | | | | at Day 27 | | | | | |
| CD | YIR037W | USP | Mito | Health | 1.2 | NS | 6 | 4 | 2 |
| | | | | Index | | | | | |
| CD | YIR037W | USP | Plastid | Biomass | −7.9 | 0.015 | 6 | 1 | 5 |
| | | | | at Day 20 | | | | | |
| CD | YIR037W | USP | Plastid | Biomass | −8.9 | 0.007 | 6 | 0 | 6 |
| | | | | at Day 27 | | | | | |
| CD | YIR037W | USP | Plastid | Health | −1.0 | NS | 6 | 1 | 5 |
| | | | | Index | | | | | |

Table 6 shows that Arabidopsis plants expressing the YIR037W gene controlled by the PCUbi promoter when targeted to the chloroplast were larger than controls under water limiting conditions, indicating better adaptation to the stress environment. In addition, the transgenic plants expressing YIR037W were darker green in color than the controls under both well-watered and water limiting conditions as shown by the increased health index. This suggests that the YIR037W transgenic plants produced more chlorophyll or had less chlorophyll degradation compared to the control plants.

When expression of gene YIR037W was controlled by the USP promoter and targeted to the chloroplast, YIR037W transgenic plants were smaller than control plants under both well-watered and water limiting conditions. Additionally, YIR037W transgenic plants were less green than control plants under well-watered conditions as shown by the decreased health index. This suggests that the YIR037W transgenic plants with this specific construct produced less chlorophyll or had more chlorophyll degradation compared to the control plants. If the targeting of YIR037W gene was the mitochondria under the control of the USP promoter, no significant difference in biomass or health index was seen when comparing YIR037W transgenic and control plants.

### H. Gamma-glutamyltranspeptidase

The gamma-glutamyltranspeptidase gene slr1269 (SEQ ID NO: 45) was expressed in Arabidopsis under control the PCUbi promoter with targeting to the chloroplast, to the mitochondria, or no subcellular targeting. Table 7 sets forth biomass and health index data obtained from the Arabidopsis plants transformed with these constructs and tested under well-watered or cycling drought conditions.

TABLE 7

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW | slr1269 | PCUbi | Mito | Biomass | 0.6 | NS | 6 | 3 | 3 |
| | | | | at Day 17 | | | | | |
| WW | slr1269 | PCUbi | Mito | Biomass | 0.2 | NS | 6 | 2 | 4 |
| | | | | at Day 21 | | | | | |
| WW | slr1269 | PCUbi | Mito | Health | −10.6 | 0.002 | 6 | 2 | 4 |
| | | | | Index | | | | | |
| WW | slr1269 | PCUbi | None | Biomass | 6.1 | 0.060 | 7 | 4 | 3 |
| | | | | at Day 17 | | | | | |
| WW | slr1269 | PCUbi | None | Biomass | 0.1 | NS | 7 | 3 | 4 |
| | | | | at Day 21 | | | | | |
| WW | slr1269 | PCUbi | None | Health | −3.1 | NS | 7 | 3 | 4 |
| | | | | Index | | | | | |
| WW | slr1269 | PCUbi | Plastid | Biomass | 4.4 | 0.056 | 6 | 4 | 2 |
| | | | | at Day 17 | | | | | |
| WW | slr1269 | PCUbi | Plastid | Biomass | 2.8 | NS | 6 | 5 | 1 |
| | | | | at Day 21 | | | | | |
| WW | slr1269 | PCUbi | Plastid | Health | −7.5 | 0.034 | 6 | 2 | 4 |
| | | | | Index | | | | | |
| CD | slr1269 | PCUbi | Mito | Biomass | −14.5 | 0.000 | 6 | 1 | 5 |
| | | | | at Day 20 | | | | | |
| CD | slr1269 | PCUbi | Mito | Biomass | −10.8 | 0.000 | 6 | 2 | 4 |
| | | | | at Day 27 | | | | | |
| CD | slr1269 | PCUbi | Mito | Health | −10.2 | 0.002 | 6 | 0 | 6 |
| | | | | Index | | | | | |
| CD | slr1269 | PCUbi | None | Biomass | 23.4 | 0.000 | 7 | 6 | 1 |
| | | | | at Day 20 | | | | | |
| CD | slr1269 | PCUbi | None | Biomass | 12.2 | 0.006 | 7 | 4 | 3 |
| | | | | at Day 27 | | | | | |
| CD | slr1269 | PCUbi | None | Health | 19.4 | 0.000 | 7 | 7 | 0 |
| | | | | Index | | | | | |
| CD | slr1269 | PCUbi | Plastid | Biomass | −4.3 | NS | 5 | 2 | 3 |
| | | | | at Day 20 | | | | | |
| CD | slr1269 | PCUbi | Plastid | Biomass | −6.8 | 0.018 | 5 | 2 | 3 |
| | | | | at Day 27 | | | | | |
| CD | slr1269 | PCUbi | Plastid | Health | −1.9 | NS | 5 | 2 | 3 |
| | | | | Index | | | | | |

Table 7 shows that Arabidopsis plants expressing the slr1269 gene targeted to the mitochondria were smaller than controls under water limiting conditions. Additionally, slr1269 transgenic plants were less green than control plants in both well-watered and water limiting conditions, as shown by the decreased health index. This suggests that the slr1269 transgenic plants with targeting to the mitochondria produced less chlorophyll or had more chlorophyll degradation compared to the control plants. Similar results were seen when expression of gene slr1269 was targeted to the chloroplast. Under water-limiting conditions, slr1269 transgenic plants were smaller than controls. Under well-watered conditions, slr1269 transgenic plants were less green than controls, as indicated by the decreased health index.

When expression of the slr1269 gene had no subcellular targeting, slr1269 transgenic plants were larger than control plants under water limiting conditions, indicating better adaptation to the stress environment. In addition, the transgenic plants expressing slr1269 were darker green in color than the controls under water limiting conditions as shown by the increased health index. This suggests that the slr1269 transgenic plants produced more chlorophyll or had less chlorophyll degradation compared to the control plants.

### G. ATP Synthase Subunit B′

The ATP synthase subunit B′ gene SLL1323 (SEQ ID NO: 47) was expressed in Arabidopsis under control the PCUbi promoter with targeting to the mitochondria. Table 8 sets forth biomass and health index data obtained from the Arabidopsis plants transformed with these constructs and tested under well-watered or cycling drought conditions.

TABLE 8

| Assay | | | | | Percent | | Valid | Positive | Negative |
|---|---|---|---|---|---|---|---|---|---|
| Type | Gene | Promoter | Target | Trait | Change | pValue | Events | Events | Events |
| WW | SLL1323 | PCUbi | Mito | Biomass at | 14.1 | 0.0001 | 6 | 5 | 1 |
| | | | | Day 17 | | | | | |
| WW | SLL1323 | PCUbi | Mito | Biomass at | 11.2 | 0.0000 | 6 | 5 | 1 |
| | | | | Day 21 | | | | | |
| WW | SLL1323 | PCUbi | Mito | Health | 2.4 | NS | 6 | 3 | 3 |
| | | | | Index | | | | | |
| CD | SLL1323 | PCUbi | Mito | Biomass at | 27.2 | 0.0000 | 6 | 6 | 0 |
| | | | | Day 20 | | | | | |
| CD | SLL1323 | PCUbi | Mito | Biomass at | 23.6 | 0.0000 | 6 | 6 | 0 |
| | | | | Day 27 | | | | | |
| CD | SLL1323 | PCUbi | Mito | Health | 6.9 | 0.0061 | 6 | 5 | 1 |
| | | | | Index | | | | | |

Table 8 shows that Arabidopsis plants expressing the SLL1323 gene resulted in plants that were larger under both well-watered and water limiting conditions. Variation does exist among transgenic plants that contain the SLL1323 gene, due to different sites of DNA insertion and other factors that impact the level or pattern of gene expression. In these experiments, the majority of independent transgenic events expressing the SLL1323 gene were larger than the controls indicating better adaptation to the stress environment. In addition, the transgenic plants expressing SLL1323 were darker green in color than the controls under water limiting conditions as shown by the increased health index. This suggests that the plants produced more chlorophyll or had less chlorophyll degradation during stress than the control plants.

### H. C-22 Sterol Desaturase

The YMR015C gene (SEQ ID NO: 51), which encodes C-22 sterol desaturase, was expressed and targeted to the chloroplast in Arabidopsis using three constructs. In one, transcription is controlled by the PCUbi promoter. In another, trancription is controlled by the Super promoter. Transcription of YMR015C in the third construct is controlled by the USP promoter. Table 9 sets forth biomass and health index data obtained from Arabidopsis plants transformed with these constructs and tested under well-watered and water-limiting conditions.

TABLE 9

| Assay | | | | | Percent | p- | Valid | Positive | Negative |
|---|---|---|---|---|---|---|---|---|---|
| Type | Gene | Promoter | Target | Measurement | Change | Value | Events | Events | Events |
| CD | YMR015C | PCUbi | Plastid | Biomass | 9.5 | 0.0150 | 6 | 4 | 2 |
| | | | | at day 20 | | | | | |
| CD | YMR015C | PCUbi | Plastid | Biomass | 17.1 | 0.0019 | 6 | 5 | 1 |
| | | | | at day 27 | | | | | |
| CD | YMR015C | PCUbi | Plastid | Health | 7.8 | 0.0416 | 6 | 4 | 2 |
| | | | | index | | | | | |
| CD | YMR015C | Super | Plastid | Biomass | 10.2 | 0.0013 | 6 | 4 | 2 |
| | | | | at day 20 | | | | | |
| CD | YMR015C | Super | Plastid | Biomass | −1.7 | NS | 6 | 2 | 4 |
| | | | | at day 27 | | | | | |
| CD | YMR015C | Super | Plastid | Health | 9.4 | 0.0003 | 6 | 4 | 2 |
| | | | | index | | | | | |
| WW | YMR015C | PCUbi | Plastid | Biomass | −16.0 | 0.0000 | 8 | 0 | 8 |
| | | | | at day 20 | | | | | |
| WW | YMR015C | PCUbi | Plastid | Biomass | −10.7 | 0.0003 | 8 | 1 | 7 |
| | | | | at day 27 | | | | | |
| WW | YMR015C | PCUbi | Plastid | Health | −8.7 | 0.0144 | 8 | 3 | 5 |
| | | | | index | | | | | |
| WW | YMR015C | Super | Plastid | Biomass | −30.8 | 0.0000 | 6 | 0 | 6 |
| | | | | at day 20 | | | | | |
| WW | YMR015C | Super | Plastid | Biomass | −20.1 | 0.0000 | 6 | 0 | 6 |
| | | | | at day 27 | | | | | |
| WW | YMR015C | Super | Plastid | Health | −13.5 | 0.0045 | 6 | 1 | 5 |
| | | | | index | | | | | |
| WW | YMR015C | USP | Plastid | Biomass | −39.5 | 0.0000 | 4 | 0 | 4 |
| | | | | at day 20 | | | | | |
| WW | YMR015C | USP | Plastid | Biomass | −28.7 | 0.0000 | 4 | 0 | 4 |
| | | | | at day 27 | | | | | |
| WW | YMR015C | USP | Plastid | Health | −16.8 | 0.0006 | 4 | 1 | 3 |
| | | | | index | | | | | |

Table 9 shows that Arabidopsis plants with the PCUbi promoter controlling expression of YMR015C were significantly larger than the control plants when the protein was also targeted to the chloroplast. In addition, these transgenic plants and those with the Super promoter controlling expression of YMR015C were darker green in color than the controls. These data indicate that the plants produced more chlorophyll or had less chlorophyll degradation during stress than the control plants. Table 9 also shows that the majority of independent transgenic events were larger than the controls.

Table 9 shows that Arabidopsis plants grown under well-watered conditions with the either the PCUbi promoter or the Super promoter controlling expression of YMR015C were significantly smaller than the control plants when the protein was also targeted to the chloroplast. Table 9 also shows that the majority of independent transgenic events were smaller than the controls. In addition, both of these constructs significantly reduced the amount of green color of the plants when grown under well-watered conditions.

1-3. (canceled)

4. A transgenic plant transformed with an expression cassette comprising, in operative association,
a) an isolated polynucleotide encoding a promoter; and
b) an isolated polynucleotide encoding a full-length hydrogenase-2 accessory protein polypeptide comprising amino acids 1 to 79 of SEQ ID NO: 16 or amino acids 1 to 82 of SEQ ID NO:16, wherein the transgenic plant demonstrates increased yield as compared to a wild type plant of the same variety which does not comprise the expression cassette.

5. A seed which is true-breeding for a transgene comprising, in operative association,
a) an isolated polynucleotide encoding a promoter; and
b) an isolated polynucleotide encoding a full-length hydrogenase-2 accessory protein polypeptide comprising amino acids 1 to 79 of SEQ ID NO:16 or amino acids 1 to 82 of SEQ ID NO: 16, wherein a transgenic plant grown from said seed demonstrates increased yield as compared to a wild type plant of the same variety which does not comprise the transgene.

6. A method for increasing yield of a plant, the method comprising the steps of:
a) transforming a plant cell with an expression cassette comprising, in operative association,
i) an isolated polynucleotide encoding a promoter; and
ii) an isolated polynucleotide encoding a full-length hydrogenase-2 accessory protein polypeptide comprising amino acids 1 to 79 of SEQ ID NO:16 or amino acids 1 to 82 of SEQ ID NO: 16;
b) regenerating transgenic plants from the transformed plant cell; and
c) selecting transgenic plants which demonstrate increased yield as compared to a wild type plant of the same variety which does not comprise the expression cassette.

7. A transgenic plant transformed with an expression cassette comprising, in operative association,
a) an isolated polynucleotide encoding a promoter; and
b) an isolated polynucleotide encoding a full-length gamma-glutamyltranspeptidase polypeptide comprising amino acids 21 to 511 of SEQ ID NO: 46,
wherein the transgenic plant demonstrates increased yield as compared to a wild type plant of the same variety which does not comprise the expression cassette.

8. The transgenic plant of claim 7, wherein the expression cassette further comprises an isolated polynucleotide encoding a chloroplast transit peptide.

9. A seed which is true-breeding for a transgene comprising, in operative association,
a) an isolated polynucleotide encoding a promoter; and
b) an isolated polynucleotide encoding a full-length gamma-glutamyltranspeptidase polypeptide comprising amino acids 21 to 511 of SEQ ID NO: 46,
wherein a transgenic plant grown from said seed demonstrates increased yield as compared to a wild type plant of the same variety which does not comprise the transgene.

10. The seed of claim 9, wherein the expression cassette further comprises an isolated polynucleotide encoding a chloroplast transit peptide.

11. A method for increasing yield of a plant, the method comprising the steps of:
a) transforming a plant cell with an expression cassette comprising, in operative association,
i) an isolated polynucleotide encoding a promoter; and
ii) an isolated polynucleotide encoding a full-length gamma-glutamyltranspeptidase polypeptide comprising amino acids 21 to 511 of SEQ ID NO: 46;
b) regenerating transgenic plants from the transformed plant cell; and
c) selecting transgenic plants which demonstrate increased yield as compared to a wild type plant of the same variety which does not comprise the expression cassette.

12. A transgenic plant transformed with an expression cassette comprising, in operative association,
a) an isolated polynucleotide encoding a promoter;
b) an isolated polynucleotide encoding a mitochondrial transit peptide; and
c) an isolated polynucleotide encoding a full-length ATP synthase subunit B′ polypeptide comprising an ATP-synt_B signature selected from the group consisting of amino acids 7 to 138 of SEQ ID NO: 48 and amino acids 82 to 213 of SEQ ID NO: 50, wherein the transgenic plant demonstrates increased yield as compared to a wild type plant of the same variety which does not comprise the expression cassette.

13. A seed which is true-breeding for a transgene comprising, in operative association,
a) an isolated polynucleotide encoding a promoter;
b) an isolated polynucleotide encoding a mitochondrial transit peptide; and
c) an isolated polynucleotide encoding a full-length ATP synthase subunit B′ polypeptide comprising an ATP-synt_B signature selected from the group consisting of amino acids 7 to 138 of SEQ ID NO: 48 and amino acids 82 to 213 of SEQ ID NO: 50, wherein a transgenic plant grown from said seed demonstrates increased yield as compared to a wild type plant of the same variety which does not comprise the transgene.

14. A method for increasing yield of a plant, the method comprising the steps of:
a) transforming a plant cell with an expression cassette comprising, in operative association,
i) an isolated polynucleotide encoding a promoter;
ii) an isolated polynucleotide encoding a mitochondrial transit peptide;
and
iii) an isolated polynucleotide encoding a full-length ATP synthase subunit B′ polypeptide comprising an ATP-synt_B signature selected from the group consisting of amino acids 7 to 138 of SEQ ID NO: 48 and amino acids 82 to 213 of SEQ ID NO: 50;
b) regenerating transgenic plants from the transformed plant cell; and
c) selecting transgenic plants which demonstrate increased yield as compared to a wild type plant of the same variety which does not comprise the expression cassette.

15. An isolated polynucleotide selected from the group consisting of:
a) a polynucleotide encoding a polypeptide having an amino acid sequence selected from the group consisting of: SEQ ID NO: 4, SEQ ID NO: 6, SEQ ID NO: 8; SEQ ID NO: 12, SEQ ID NO: 14, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 50, and SEQ ID NO: 54; and
b) a polynucleotide having a sequence selected from the group consisting of: SEQ ID NO: 3, SEQ ID NO: 5, SEQ ID NO: 7, SEQ ID NO: 11, SEQ ID NO: 13, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43. SEQ ID NO: 49, and SEQ ID NO: 53
